(19) United States
(12) Patent Application Publication
(10) Pub. No.: US 2023/0248665 A1
(43) Pub. Date: Aug. 10, 2023

(54) COMPOSITION COMPRISING DIMETHYLCHALCONE DERIVATIVE FOR PREVENTION OR TREATMENT OF METABOLIC DISEASE

(71) Applicants: SEOUL NATIONAL UNIVERSITY R & DB FOUNDATION, Seoul (KR); SEOUL NATIONAL UNIVERSITY HOSPITAL, Seoul (KR); CHUNG-ANG UNIVERSITY INDUSTRY-ACADEMIC COOPERATION FOUNDATION, Seoul (KR)

(72) Inventors: Kyong Soo PARK, Seoul (KR); Sung Soo CHUNG, Seoul (KR); Ji Seon LEE, Seoul (KR); Kwang Yong PARK, Seoul (KR)

(73) Assignees: SEOUL NATIONAL UNIVERSITY R & DB FOUNDATION, Seoul (KR); SEOUL NATIONAL UNIVERSITY HOSPITAL, Seoul (KR); CHUNG-ANG UNIVERSITY INDUSTRY-ACADEMIC COOPERATION FOUNDATION, Seoul (KR)

(21) Appl. No.: 18/185,061

(22) Filed: Mar. 16, 2023

Related U.S. Application Data
(63) Continuation-in-part of application No. PCT/KR2021/012820, filed on Sep. 17, 2021.

(30) Foreign Application Priority Data
Sep. 18, 2020 (KR) 10-2020-0120378
Sep. 16, 2021 (KR) 10-2021-0124071

(51) Int. Cl. A61K 31/12; A61P 3/10; A61P 9/10

(52) U.S. Cl. CPC A61K 31/12; A61P 3/10; A61P 9/10

(57) ABSTRACT

Provided is a composition comprising a dimethylchalcone derivative for prevention or treatment of metabolic diseases and, more specifically, to a compositioncomprising a derivative of 2′,4′-dihydroxy-6′-methoxy-3′,5′-dimethylchalcone as an active ingredient for the alleviation, prevention, or treatment of various metabolic diseases.

### CROSS-REFERENCE TO RELATED APPLICATION

This application is a Continuation-in-Part of PCT/KR2021/012820 filed on Sep. 17, 2021, which claims the benefit of priority from Korean Patent Application No. 10-2020-0120378 filed on Sep. 18, 2020 and Korean Patent Application No. 10-2021-0124071 filed on Sep. 16, 2021, the contents of each of which are incorporated herein by reference in its entirety.

### BACKGROUND

### 1. Field of the Invention

The present invention relates to a composition comprising a dimethylchalcone derivative for prevention or treatment of metabolic diseases and, more specifically, to a composition comprising a derivative of 2′,4′-dihydroxy-6′-methoxy-3′,5′-dimethylchalcone as an active ingredient for the alleviation, prevention, or treatment of metabolic diseases.

### 2. Discussion of Related Art

A metabolic disease refers to a disease in which various diseases such as obesity, diabetes, hypertension, hyperlipidemia, coronary or arteriosclerosis, and non-alcoholic fatty liver occur simultaneously due to chronic metabolic disorders, and it was first identified by Reaven in 1988 (Korean Patent Application Laid-Open No. 10-2015-0054439). Metabolic diseases are characterized by insulin resistance, hypertension, dyslipidemia, and the like, and most of them are accompanied by overweight or obesity. The most serious problem of metabolic diseases are the occurrence of chronic complications such as diabetic retinopathy, diabetic nephropathy, diabetic foot disease, diabetic neuropathy, hyperlipidemia, and cardiovascular disease (stroke, angina pectoris, myocardial infarction, peripheral vascular disease). Most of these chronic complications go through an irreversible process once they occur, and since there is no method capable of completely blocking such a process, these chronic complications cause serious symptoms and even lead to the death of a patient when not appropriately treated. However, patients have taken hypoglycemic agents, antihypertensive agents, cholesterol-lowering agents, and the like individually to date in order to treat metabolic diseases with these complex symptoms. Therefore, there is a need for developing a novel therapeutic agent capable of simultaneously treating various symptoms in order to effectively manage and treat metabolic diseases with these complex symptoms.

### SUMMARY OF THE INVENTION

The present invention has been made in an effort to solve the aforementioned problems in the related art, and an object thereof is to provide a composition comprising a derivative of 2′,4′-dihydroxy-6′-methoxy-3′,5′-dimethylchalcone capable of simultaneously treating the complex symptoms of metabolic diseases as an active ingredient for the alleviation, prevention, or treatment of metabolic diseases.

However, the technical problems which the present invention intends to solve are not limited to the technical problems which have been mentioned above, and other technical problems which have not been mentioned will clearly be understood by those with ordinary skill in the art to which the present invention pertains from the following description.

The present invention provides a composition comprising a derivative of 2′,4′-dihydroxy-6′-methoxy-3′,5′-dimethylchalcone (DMC) represented by the following Chemical Formula 1, or a pharmaceutically acceptable salt thereof as an active ingredient for the alleviation, prevention, or treatment of metabolic diseases. The composition may be preferably a pharmaceutical composition, a food composition, a functional food composition, and the like, but is not limited thereto.

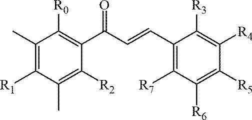

In Chemical Formula 1, R₀, R₁, and R₂ are each independently a hydroxy group (OH), a methoxymethoxy group (OCH₂OCH₃; OMOM), or a C1-C10 alkoxy group,
R₃ to R₇ are each independently any one selected from the group consisting of a halogen element, hydrogen (H), deuterium (D), a hydroxy group (OH), a thiol group (SH), an amino group (NH₂), a nitrile group, a nitro group, a substituted or unsubstituted C1-C10 alkylamino group, a substituted or unsubstituted C1-C10 alkoxy group, a substituted or unsubstituted C1-C10 alkylsulfoxy group, a substituted or unsubstituted C1-C10 alkyl group, a substituted or unsubstituted C2-C10 alkenyl, a substituted or unsubstituted C2-C10 alkynyl, a substituted or unsubstituted C6-C20 aryl group, a substituted or unsubstituted C1-C10 alkylthio group, and a substituted or unsubstituted C1-C10 alkylsulfonyl group,
the ‘substituted or unsubstituted’ is unsubstituted or substituted with one or more substituents selected from the group consisting of a halogen group, a nitrile group, a nitro group, a hydroxy group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an alkynyl group, an aryl group, an aralkyl group, an aralkenyl group, an alkylaryl group, an alkylamine group, an aralkylamine group, a heteroarylamine group, an arylamine group, an arylphosphine group, and a heterocyclic group.

In an exemplary embodiment of the present invention, at least any one of R₀, R₁, and R₂ may not be OH, but is not limited thereto.

Further, in an exemplary embodiment of the present invention, when R₀ and R₁ are simultaneously a methoxy group (OCH₃; OMe) or simultaneously OMOM, R₂ may not be OH or OMOM, but is not limited thereto.

In addition, in an exemplary embodiment of the present invention, R₆ and R₇ may each be independently H, OH, a C1-C5 alkoxy group, or OMOM, but are not limited thereto. More preferably, R₆ and R₇ may each be independently H, OH, OMe, or OMOM.

Furthermore, in an exemplary embodiment of the present invention, when R₁ and R₂ are simultaneously a hydroxy group, at least any one of R₃ to R₇ may not be hydrogen, but is not limited thereto.

Further, in an exemplary embodiment of the present invention, when R₁ and R₂ are simultaneously a hydroxy group, R₃ to R₇ may not be a hydroxy group, but are not limited thereto.

In addition, in an exemplary embodiment of the present invention, the alkylthio group may be a C1-C5 alkylthio group, and may be more preferably a methylthio group or an ethylthio group, but is not limited thereto.

In the present invention, the halogen element is a Group 17 element of the Periodic Table, and includes fluorine (F), chlorine (Cl), bromine (Br), iodine (I), and the like.

In the present specification, the term “C1-C10” includes C1-C10, C1-C8, C1-C5, C1-C3, C1-C2, and the like. Likewise, C2-C10 includes C2-C10, C2-C8, C2-C6, C2-C5, C2-C4, C2-C3, and the like. C6-C20 includes C6-C18, C6-C15, C6-C12, C6-C10, C6-C8, and the like.

In an exemplary embodiment of the present invention, the derivative of the 2′,4′-dihydroxy-6′-methoxy-3′,5′-dimethylchalcone represented by Chemical Formula 1 may be preferably any one or more compounds selected from the group consisting of the following Chemical Formulae 2 to 39 or a pharmaceutically acceptable salt thereof, but is not limited as long as it is a derivative derived from 2′,4′-dihydroxy-6′-methoxy-3′,5′-dimethylchalcone. Throughout the specification, the ‘compound represented by Chemical Formula N’ may be simply referred to as ‘Compound N’.

| | | | |
|---|---|---|---|
| Chemical formula 2 | Chemical formula 3 | Chemical formula 4 | Chemical formula 5 |
| 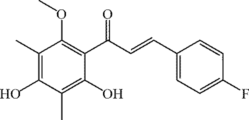 [SMILES: COc1c(C)c(O)c(C)c(O)c1C(=O)/C=C/c1ccc(F)cc1] | 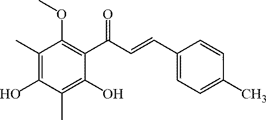 [SMILES: COc1c(C)c(O)c(C)c(O)c1C(=O)/C=C/c1ccc(C)cc1] | 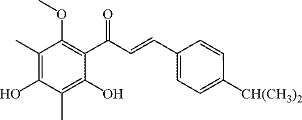 [SMILES: COc1c(C)c(O)c(C)c(O)c1C(=O)/C=C/c1ccc(C(C)C)cc1] | 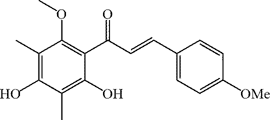 [SMILES: COc1ccc(/C=C/C(=O)c2c(O)c(C)c(O)c(C)c2OC)cc1] |
| Chemical formula 6 | Chemical formula 7 | Chemical formula 8 | Chemical formula 9 |
| 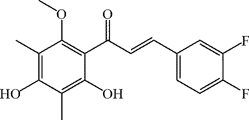 [SMILES: COc1c(C)c(O)c(C)c(O)c1C(=O)/C=C/c1ccc(F)c(F)c1] | 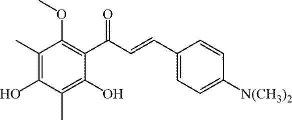 [SMILES: COc1c(C)c(O)c(C)c(O)c1C(=O)/C=C/c1ccc(N(C)C)cc1] | 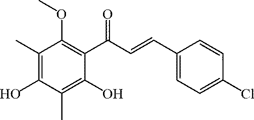 [SMILES: COc1c(C)c(O)c(C)c(O)c1C(=O)/C=C/c1ccc(Cl)cc1] | 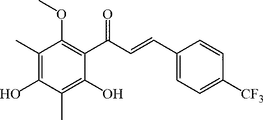 [SMILES: COc1c(C)c(O)c(C)c(O)c1C(=O)/C=C/c1ccc(C(F)(F)F)cc1] |
| Chemical formula 10 | Chemical formula 11 | Chemical formula 12 | Chemical formula 13 |
| 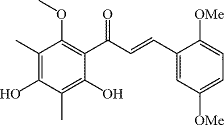 [SMILES: COc1ccc(OC)c(/C=C/C(=O)c2c(O)c(C)c(O)c(C)c2OC)c1] | 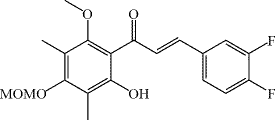 | 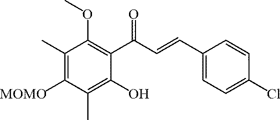 | 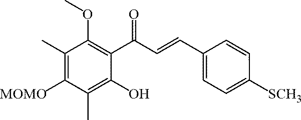 |
| Chemical formula 14 | Chemical formula 15 | Chemical formula 16 | Chemical formula 17 |
| 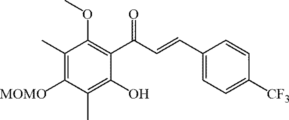 | 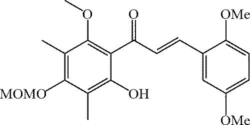 | 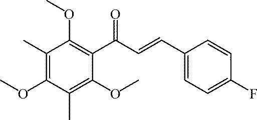 [SMILES: COc1c(C)c(OC)c(C(=O)/C=C/c2ccc(F)cc2)c(OC)c1C] | 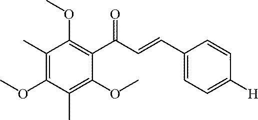 [SMILES: COc1c(C)c(OC)c(C(=O)/C=C/c2ccccc2)c(OC)c1C] |
| Chemical formula 18 | Chemical formula 19 | Chemical formula 20 | Chemical formula 21 |
| 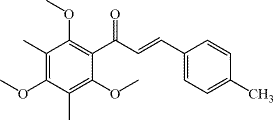 | 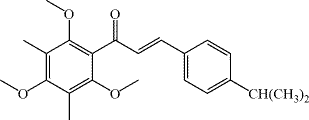 [SMILES: COc1c(C)c(OC)c(C(=O)/C=C/c2ccc(C(C)C)cc2)c(OC)c1C] | 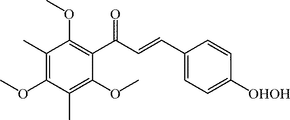 | 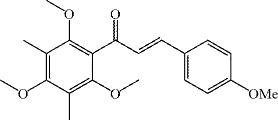 [SMILES: COc1ccc(/C=C/C(=O)c2c(OC)c(C)c(OC)c(C)c2OC)cc1] |
| Chemical formula 22 | Chemical formula 23 | Chemical formula 24 | Chemical formula 25 |
| 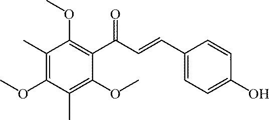 [SMILES: COc1c(C)c(OC)c(C(=O)/C=C/c2ccc(O)cc2)c(OC)c1C] | 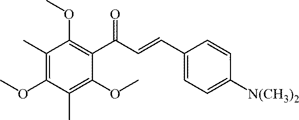 [SMILES: COc1c(C)c(OC)c(C(=O)/C=C/c2ccc(N(C)C)cc2)c(OC)c1C] | 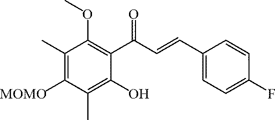 | 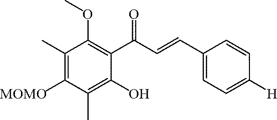 |
| Chemical formula 26 | Chemical formula 27 | Chemical formula 28 | Chemical formula 29 |
| 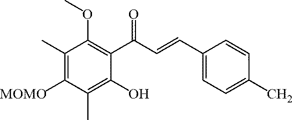 | 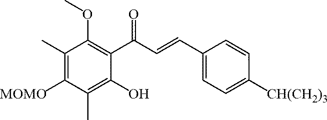 | 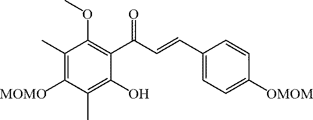 | 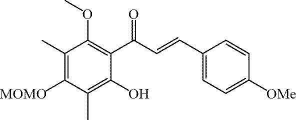 |
| Chemical formula 30 | Chemical formula 31 | Chemical formula 32 | Chemical formula 33 |
| 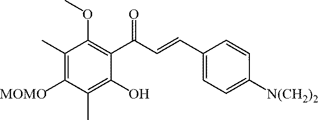 | 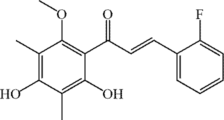 [SMILES: COc1c(C)c(O)c(C)c(O)c1C(=O)/C=C/c1ccccc1F] | 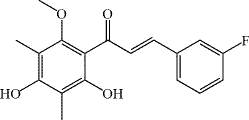 [SMILES: COc1c(C)c(O)c(C)c(O)c1C(=O)/C=C/c1cccc(F)c1] | 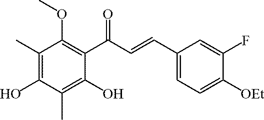 [SMILES: CCOc1ccc(/C=C/C(=O)c2c(O)c(C)c(O)c(C)c2OC)cc1F] |
| Chemical formula 34 | Chemical formula 35 | Chemical formula 36 | Chemical formula 37 |
| 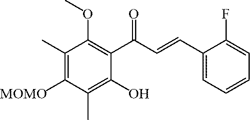 | 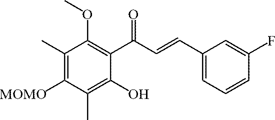 | 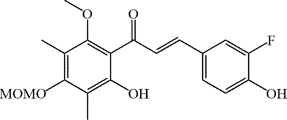 | 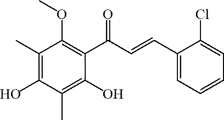 [SMILES: COc1c(C)c(O)c(C)c(O)c1C(=O)/C=C/c1ccccc1Cl] |
| Chemical formula 38 | Chemical formula 39 | | |
| 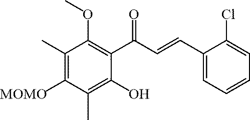 | 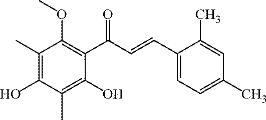 [SMILES: COc1c(C)c(O)c(C)c(O)c1C(=O)/C=C/c1ccc(C)cc1C] | | |

In another exemplary embodiment of the present invention, the metabolic disease may be preferably any one or more selected from the group consisting of obesity, diabetes, hypertension, hyperlipidemia, arteriosclerosis, coronary arteriosclerosis, non-alcoholic fatty liver, diabetic retinopathy, diabetic nephropathy, diabetic foot disease, diabetic neuropathy, stroke, angina pectoris, myocardial infarction, and peripheral vascular disease, and is not limited thereto as long as it is a metabolic disease which can be treated by increasing the activity of PPARy and/or AMPK, or increasing the fatty acid oxidation rate in muscle, or suppressing the migration of vascular smooth muscle cells to the tunica intima, or exhibiting an antioxidant effect, or activating the Nrf2 protein.

Further, the present invention provides a method for preventing, alleviating, or treating metabolic diseases, the method including administering a composition comprising the derivative of the 2′,4′-dihydroxy-6′-methoxy-3′,5′-dimethylchalcone represented by Chemical Formula 1 or a pharmaceutically acceptable salt thereof as an active ingredient to a subject in need thereof.

In addition, the present invention provides a use of a composition comprising the derivative of the 2′,4′-dihydroxy-6′-methoxy-3′,5′-dimethylchalcone or a pharmaceutically acceptable salt thereof as an active ingredient for the alleviation, prevention, or treatment of metabolic diseases.

Furthermore, the present invention provides a use of the derivative of the 2′,4′-dihydroxy-6′-methoxy-3′,5′-dimethylchalcone or a pharmaceutically acceptable salt thereof for producing a drug used for metabolic diseases.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a view illustrating the results of confirming the effect of DMC derivatives according to an exemplary embodiment of the present invention on PPARy activity;

FIG. 2 is a view illustrating the results of confirming the effect of DMC derivatives according to an exemplary embodiment of the present invention on AMPK activity;

FIG. 3 is a view illustrating the results of confirming the effect of DMC derivatives according to an exemplary embodiment of the present invention on fatty acid oxidation;

FIG. 4 is a set of views illustrating the results of confirming the effect of DMC derivatives according to an exemplary embodiment of the present invention on the migration of vascular smooth muscle cells;

FIG. 5 is a view illustrating the results of confirming the effect of DMC derivatives according to an exemplary embodiment of the present invention on the binding of vascular endothelial cells and immune cells;

FIG. 6 is a view illustrating the results of confirming the antioxidant effect of DMC derivatives according to an exemplary embodiment of the present invention in kidney cells;

FIG. 7 is a view illustrating the results of confirming the effect of a DMC derivative according to an exemplary embodiment of the present invention on Nrf2 protein expression levels in kidney cells;

FIG. 8 is a view illustrating the results of confirming the antioxidant effect of a DMC derivative according to an exemplary embodiment of the present invention in hepatocytes;

FIG. 9 is a view illustrating the results of confirming the effect of a DMC derivative according to an exemplary embodiment of the present invention on Nrf2 protein expression levels in hepatocytes;

FIG. 10 is a view illustrating the results of confirming the effect of DMC derivatives according to an exemplary embodiment of the present invention on an increase in body weight caused by a high-fat diet;

FIG. 11 is a view illustrating the results of confirming the glucose tolerance effect of DMC derivatives according to an exemplary embodiment of the present invention;

FIG. 12 is a view illustrating the results of confirming the effect of DMC derivatives according to an exemplary embodiment of the present invention on fatty acid oxidation in muscle; and

FIG. 13 is a view illustrating the results of confirming the effect of DMC derivatives according to an exemplary embodiment of the present invention on fat accumulation in liver tissue.

FIG. 14 is a view illustrating the results of confirming whether DMC derivatives according to an exemplary embodiment of the present invention has cytotoxicity.

### DETAILED DESCRIPTION OF EXEMPLARY EMBODIMENTS

The 2′,4′-dihydroxy-6′-methoxy-3′,5′-dimethylchalcone derivatives of the present invention not only may lower insulin resistance by increasing the activity of PPARy and AMPK, but also may increase the fatty acid oxidation rate in muscle. Further, the derivatives may prevent blood vessels from narrowing by suppressing the migration of vascular smooth muscle cells to the tunica intima and binding of vascular endothelial cells and immune cells. In addition, the DMC derivatives serve as the activating agents of Nrf2 to exhibit an antioxidant effect. Therefore, the derivatives of the present invention may effectively treat not only various metabolic diseases showing complex symptoms but also complications caused by the metabolic diseases.

Therefore, the present invention provides a derivative of 2′,4′-dihydroxy-6′-methoxy-3′,5′-dimethylchalcone represented by the following Chemical Formula 1 or a pharmaceutically acceptable salt thereof; and a use of the same for the prevention or treatment of metabolic diseases:

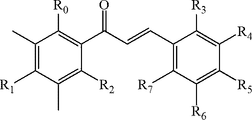

wherein, in Chemical Formula 1,
R₀, R₁, and R₂ are each independently a hydroxy group (OH), a methoxymethoxy group (OCH₂OCH₃; OMOM), or a C1-C10 alkoxy group, wherein at least any one of R₀, R₁, and R₂ is not OH,
when R₀ and R₁ are simultaneously a methoxy group (OCH₃; OMe) or simultaneously OMOM, R₂ is not OH or OMOM,
R₃ to R₇ are each independently any one selected from the group consisting of a halogen element, hydrogen (H), deuterium (D), a hydroxy group (OH), a thiol group (SH), an amino group (NH₂), a nitrile group, a nitro group, a substituted or unsubstituted C1-C10 alkylamino group, a substituted or unsubstituted C1-C10 alkoxy group, a substituted or unsubstituted C1-C10 alkylsulfoxy group, a substituted or unsubstituted C1-C10 alkyl group, a substituted or unsubstituted C2-C10 alkenyl, a substituted or unsubstituted C2-C10 alkynyl, a substituted or unsubstituted C6-C20 aryl group, a substituted or unsubstituted C1-C10 alkylthio group, and a substituted or unsubstituted C1-C10 alkylsulfonyl group,
the ‘substituted or unsubstituted’ is unsubstituted or substituted with one or more substituents selected from the group consisting of a halogen group, a nitrile group, a nitro group, a hydroxy group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an alkynyl group, an aryl group, an aralkyl group, an aralkenyl group, an alkylaryl group, an alkylamine group, an aralkylamine group, a heteroarylamine group, an arylamine group, an arylphosphine group, and a heterocyclic group,
at least any one of R₃ to R₇ is not hydrogen when R₁ and R₂ are simultaneously a hydroxy group, and
R₃ to R₇ are not a hydroxy group when R₁ and R₂ are simultaneously a hydroxy group.

As used herein, the metabolic disease is a general term for a disease in which various diseases such as obesity, diabetes, hypertension, hyperlipidemia, arteriosclerosis, coronary arteriosclerosis, and non-alcoholic fatty liver caused by chronic metabolic disorders occur in a complex manner and complications caused thereby, and examples of the complications caused thereby include diabetic retinopathy, diabetic nephropathy, diabetic foot disease, diabetic neuropathy and the like, which are diabetic complications, and hyperlipidemia, stroke, angina pectoris, myocardial infarction, peripheral vascular disease, and the like, which are cardiovascular diseases, but there is no limitation to all diseases caused by metabolic diseases.

As used herein, “insulin resistance” refers to a phenomenon that generally occurs prior to the onset of diabetes mellitus, and obesity is known as a major factor to induce insulin resistance. An increase in blood fatty acids, which occurs in obese patients, causes fat accumulation in the liver and muscles, as well as lipotoxicity, ultimately inducing insulin resistance. A tissue in which fatty acid oxidation most often occurs is muscle, and accordingly, if the ability of muscle, which occupies a large proportion of the body, to oxidize fatty acids can be increased, obesity and insulin resistance may be reduced, and ultimately the onset of diabetes may be suppressed.

As used herein, PPARγ is a transcriptional regulatory factor which is highly expressed in adipocytes and is essential for adipocyte differentiation. When PPARγ activity is increased, fat accumulation in adipose tissue is increased, so that there is an effect of reducing blood fatty acids, and increased PPARγ activity increases the expression of adipokines such as adiponectin in adipose tissue, which serves to increase the ability of muscle to oxidize fatty acids. Due to functions as described above, thiazolidinedione (TZD)-based drugs, which are well-known as PPARγ activators, are widely used as therapeutic agents for insulin-resistant diseases and diabetes.

As used herein, AMPK is a major regulator of energy metabolism, and its activity is known to be increased while Thr172 of an α-subunit is phosphorylated when energy is insufficient. AMPK phosphorylates acetyl-CoA carboxylase (ACC) to suppress the activity of ACC. It is known that ACC is an enzyme that promotes malonyl-CoA synthesis, malonyl-CoA acts as an inhibitor of carnitine palmitoyltransferase 1 (CPT1), which is a major enzyme for fatty acid oxidation, and an increase in AMPK activity ultimately increases the activity of CPT1. Therefore, AMPK has been actively studied as an important target in diabetes treatment.

Nrf2 is a protein that plays an important role in maintaining the homeostasis of cells by regulating the basal expression levels or induced expression levels of proteins which suppress oxidation when cells are exposed to chemicals or oxidative stress, or proteins related to the transport of detoxification enzymes or xenobiotics. In addition, the Nrf2 protein is also known to be involved in inflammatory responses, lipid synthesis, and the like. Keap1 binds to Nrf2 to induce the Nrf2 proteolysis by proteasomes. Conversely, it is known that Nrf2 activators separate Nrf2 from Keap1, and the separated Nrf2 translocates to the nucleus to increase the expression of antioxidant proteins such as NQO1 and HO1, thereby effectively suppressing obesity, non-alcoholic fatty liver, diabetic complications, and the like.

In the present specification, arteriosclerosis is caused by the formation of plaques in which many layers of fat are accumulated on arterial blood vessel walls, and clots of cholesterol, immune cells, vascular smooth muscle cells and secretions of these cells are included in these plaques. Arteriosclerosis progresses in stages. In the early stage, endothelial cells express adhesion molecules due to various environmental factors, and as a result, adhesion of monocytes increases, monocytes migrate to the intimal layer and differentiate into macrophages. Thereafter, macrophages are transformed into foam cells through oxLDL uptake, and macrophages and T lymphocytes in the foam cell layer secrete inflammatory cytokines, thereby causing the migration of vascular smooth muscle cells to the tunica intima. Finally, the vascular smooth muscle cells that have migrated to the blood vessels thicken the vascular wall through replication and secretion of the extracellular matrix and the like to form plaques, thrombi, and the like, thereby inducing arteriosclerosis.

In the present specification, the dimethylchalcone (DMC) derivative is not limited as long as it is a derivative derived from dimethylchalcone, but may be preferably a dimethylchalcone derivative having a multi-substituted chalcone structure using phloroglucinol as a starting material.

As used herein, the pharmaceutical acceptable salt refers to any organic or inorganic addition salt of the compounds of the present invention, whose effective concentration is non-toxic and harmless and whose side effects caused by the salt do not degrade the beneficial efficacy of the compounds of the present invention. These salts may use an inorganic acid and an organic acid as a free acid, and as the inorganic acid, it is possible to use hydrochloric acid, bromic acid, nitric acid, sulfuric acid, perchloric acid, phosphoric acid, and the like, and as the organic acid, it is possible to use citric acid, acetic acid, lactic acid, maleic acid, fumaric acid, gluconic acid, methanesulfonic acid, glycolic acid, succinic acid, tartaric acid, galacturonic acid, embonic acid, glutamic acid, aspartic acid, oxalic acid, (D) or (L) malic acid, maleic acid, methanesulfonic acid, ethanesulfonic acid, 4-toluenesulfonic acid, salicylic acid, citric acid, benzoic acid, malonic acid, and the like. Furthermore, these salts may include alkali metal salts (sodium salts, potassium salts, and the like), alkaline earth metal salts (calcium salts, magnesium salts, and the like), and the like. For example, an acid addition salt includes an acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methyl sulfate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate, aluminum, arginine, benzathine, calcium, choline, diethylamine, diolamine, glycine, lysine, magnesium, meglumine, olamine, potassium, sodium, tromethamine, zinc salts, and the like, but is not limited thereto as long as the acid addition salt can be added without affecting the efficacy of the compounds of the present invention.

As used herein, “prevention” refers to all actions that suppress metabolic diseases or delay the onset of the metabolic diseases through administration of the composition according to the present invention.

As used herein, “treatment” refers to all actions in which symptoms of metabolic diseases are ameliorated or beneficially changed by administering the composition according to the present invention.

As used herein, “alleviation” refers to all actions that at least reduce a parameter associated with a condition to be treated, for example, the degree of symptoms. In this case, the composition of the present invention may be used simultaneously with or separately from a therapeutic drug for the prevention or alleviation of metabolic diseases.

As used herein, “subject” refers to a subject to whom the composition of the invention can be administered, preferably a patient who has a metabolic disease, but is not limited to such a target.

As used herein, a pharmaceutical composition may be in the form of a capsule, a tablet, a granule, an injection, an ointment, a powder, or a beverage, and the pharmaceutical composition may be characterized in that it targets humans. The pharmaceutical composition of the present invention may include a pharmaceutically acceptable carrier. As the pharmaceutically acceptable carrier, a binder, a lubricant, a disintegrant, an excipient, a solubilizing agent, a dispersing agent, a stabilizer, a suspending agent, a colorant, a flavoring agent, and the like may be used when orally administered, in the case of injection, a buffering agent, a preservative, an analgesic, a solubilizer, an isotonic agent, a stabilizer, and the like may be mixed and used, and in the case of topical administration, a base, an excipient, a lubricant, a preservative, and the like may be used. The formulation of the pharmaceutical composition of the present invention may be variously prepared by mixing the pharmaceutical composition of the present invention with the pharmaceutically acceptable carrier as described above. For example, the formulation may be prepared in the form of a tablet, a troche, a capsule, an elixir, a suspension, a syrup, a wafer, and the like when orally administered, and in the case of injection, the injection may be formulated into unit dosage ampoules or in multiple dosage forms. The pharmaceutical composition of the present invention may also be used by being formulated into other sugar-coated tablets, gels, pills, powders, granules, suppositories, external preparations, solutions, suspensions, sustained-release preparations, slurries, and the like. Meanwhile, as an example of suitable carriers, excipients and diluents for formulation, it is possible to use lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate, mineral oil, or the like. Further, the pharmaceutical composition of the present invention may further include a filler, an anticoagulant, a lubricant, a wetting agent, a flavoring agent, an emulsifying agent, an antiseptic, and the like.

The route of administration of the pharmaceutical composition according to the present invention is not limited to the following routes, but oral or parenteral administration is preferred, and for example, oral, intravenous, intramuscular, intraarticular, intrasynovial, intraarterial, intramedullary, intrathecal, intracardiac, percutaneous, intradermal, subcutaneous, intraperitoneal, intranasal, intestinal, topical, sublingual, rectal, intrasternal, intralesional, intracranial administration, and the like are included.

The dosage of the pharmaceutical composition of the present invention may vary depending on various factors including the activity of the specific compound used, age, body weight, general health, sex, diet, time of administration, route of administration, rate of excretion, drug combination and the severity of the particular disease to be prevented or treated, and varies depending on the condition, body weight, and the degree of disease of the patient, the form of drug, the route of administration and duration, but may be appropriately selected by a person skilled in the art, and may be 0.0001 to 500 mg/kg or 0.001 to 500 mg/kg daily. The administration may be carried out once daily, or may be divided into several times. The dosage is not intended to limit the scope of the present invention in any way.

In the present specification, the food composition may be used in various foods, for example, beverages, gums, teas, vitamin complexes, and health supplements, and may be used in the form of a pill, a powder, a granule, an infusum, a tablet, a capsule, or a beverage. The food composition includes a health functional food composition. In this case, the amount of the DMC derivative of the present invention in a food or a beverage may be 0.01 to 30 wt% based on the weight of the entire food in the case of the food composition of the present invention, and in the case of the health beverage composition, the DMC derivative may be added at a proportion of 0.02 to 10 g, preferably 0.3 to 1 g, based on 100 mL.

The food composition of the present invention has no particular limitation on an ingredient to be added other than the DMC derivative of the present invention as an essential ingredient, and may include typical food additives used in the art, for example, natural carbohydrates, flavorants, flavoring agents, colorants, fillers, stabilizers, various nutrients, vitamins, minerals (electrolytes) and the like. As an example of the natural carbohydrates, typical sugars such as monosaccharides, for example, glucose, fructose and the like; disaccharides, for example, maltose, sucrose and the like; and polysaccharides, for example, dextrin, cyclodextrin and the like, and sugar alcohols such as xylitol, sorbitol, and erythritol may be used. As an example of the flavorant, a natural flavorant (thaumatin, stevia extract (for example, rebaudioside A, glycyrrhizin and the like), and a synthetic flavorant (saccharin, aspartame and the like) may be advantageously used. As an example of the flavoring agent, honey, D-mannitol, a maltitol solution, a krill concentrate, and the like may be used. In addition to those, the food composition of the present invention may contain pectic acid and salts thereof, alginic acid and salts thereof, organic acids, protective colloid thickeners, pH adjusters, stabilizers, preservatives, glycerin, alcohols, carbonating agents used in carbonated drinks, and the like. These ingredients may be used either alone or in combinations thereof. The proportion of these additives is not significantly important, but is generally selected within a range of 0 to 20 parts by weight per 100 parts by weight of the composition of the present invention.

Hereinafter, preferred examples for helping with understanding of the present invention will be suggested. However, the following examples are provided only so that the present invention may be more easily understood, and the content of the present invention is not limited by the following examples.

### Examples

### Example 1: Preparation of 2′,4′-dihydroxy-6′-methoxy-3′,5′-dimethylchalcone Derivatives

Derivatives of 2′,4′-dihydroxy-6′-methoxy-3′,5′-dimethylchalcone in the following Table 1 were synthesized.

TABLE 1

| | | | |
|---|---|---|---|
| Chemical formula 2 | Chemical formula 3 | Chemical formula 4 | Chemical formula 5 |
| 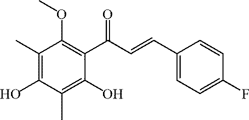 [SMILES: COc1c(C)c(O)c(C)c(O)c1C(=O)/C=C/c1ccc(F)cc1] | 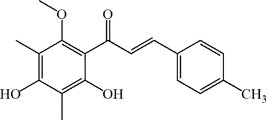 [SMILES: COc1c(C)c(O)c(C)c(O)c1C(=O)/C=C/c1ccc(C)cc1] | 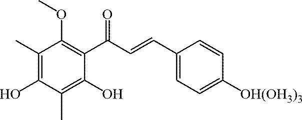 | 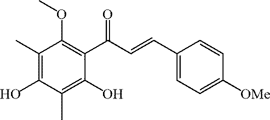 [SMILES: COc1ccc(/C=C/C(=O)c2c(O)c(C)c(O)c(C)c2OC)cc1] |
| Chemical formula 6 | Chemical formula 7 | Chemical formula 8 | Chemical formula 9 |
| 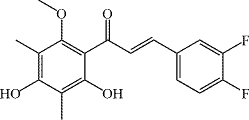 [SMILES: COc1c(C)c(O)c(C)c(O)c1C(=O)/C=C/c1ccc(F)c(F)c1] | 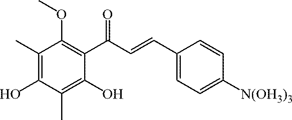 | 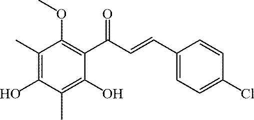 [SMILES: COc1c(C)c(O)c(C)c(O)c1C(=O)/C=C/c1ccc(Cl)cc1] | 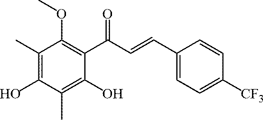 [SMILES: COc1c(C)c(O)c(C)c(O)c1C(=O)/C=C/c1ccc(C(F)(F)F)cc1] |
| Chemical formula 10 | Chemical formula 11 | Chemical formula 12 | Chemical formula 13 |
| 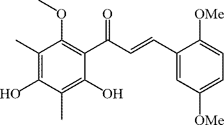 [SMILES: COc1ccc(OC)c(/C=C/C(=O)c2c(O)c(C)c(O)c(C)c2OC)c1] | 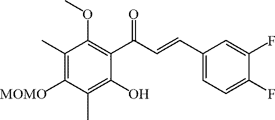 | 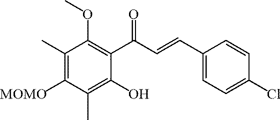 | 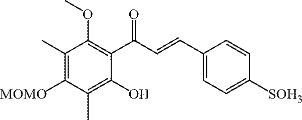 |
| Chemical formula 14 | Chemical formula 15 | Chemical formula 16 | Chemical formula 17 |
| 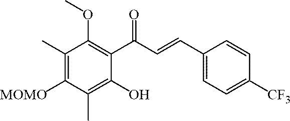 | 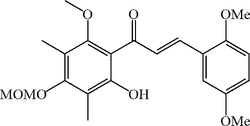 | 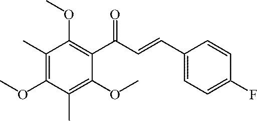 [SMILES: COc1c(C)c(OC)c(C(=O)/C=C/c2ccc(F)cc2)c(OC)c1C] | 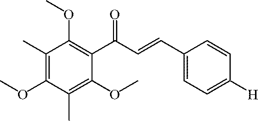 [SMILES: COc1c(C)c(OC)c(C(=O)/C=C/c2ccccc2)c(OC)c1C] |
| Chemical formula 18 | Chemical formula 19 | Chemical formula 20 | Chemical formula 21 |
| 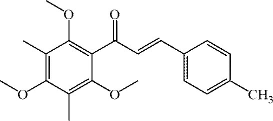 | 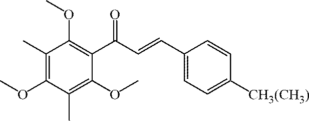 | 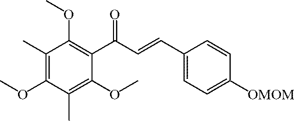 | 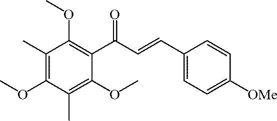 [SMILES: COc1ccc(/C=C/C(=O)c2c(OC)c(C)c(OC)c(C)c2OC)cc1] |
| Chemical formula 22 | Chemical formula 23 | Chemical formula 24 | Chemical formula 25 |
| 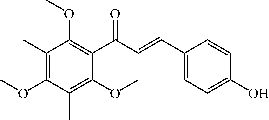 [SMILES: COc1c(C)c(OC)c(C(=O)/C=C/c2ccc(O)cc2)c(OC)c1C] | 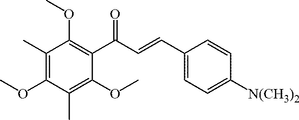 [SMILES: COc1c(C)c(OC)c(C(=O)/C=C/c2ccc(N(C)C)cc2)c(OC)c1C] | 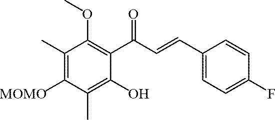 | 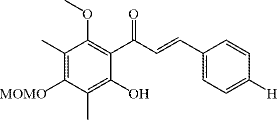 |
| Chemical formula 26 | Chemical formula 27 | Chemical formula 28 | Chemical formula 29 |
| 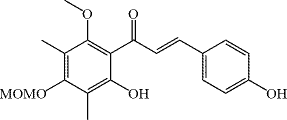 | 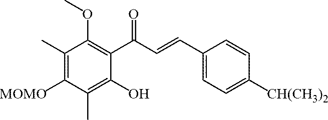 | 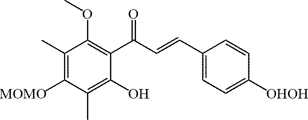 | 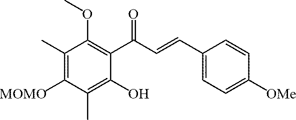 |
| Chemical formula 30 | Chemical formula 31 | Chemical formula 32 | Chemical formula 33 |
| 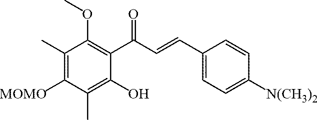 | 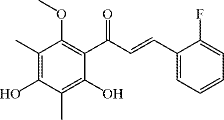 [SMILES: COc1c(C)c(O)c(C)c(O)c1C(=O)/C=C/c1ccccc1F] | 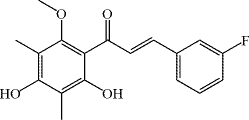 [SMILES: COc1c(C)c(O)c(C)c(O)c1C(=O)/C=C/c1cccc(F)c1] | 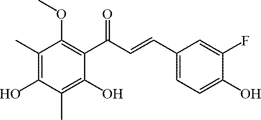 [SMILES: COc1c(C)c(O)c(C)c(O)c1C(=O)/C=C/c1ccc(O)c(F)c1] |
| Chemical formula 34 | Chemical formula 35 | Chemical formula 36 | Chemical formula 37 |
| 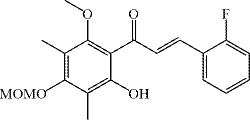 | 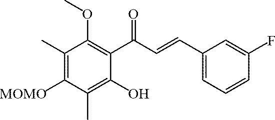 | 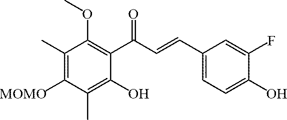 | 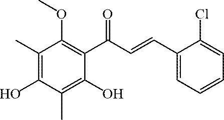 [SMILES: COc1c(C)c(O)c(C)c(O)c1C(=O)/C=C/c1ccccc1Cl] |
| Chemical formula 38 | Chemical formula 39 | | |
| 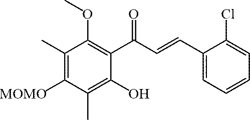 | 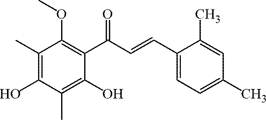 [SMILES: COc1c(C)c(O)c(C)c(O)c1C(=O)/C=C/c1ccc(C)cc1C] | | |

Reaction Scheme 1 for preparing compounds represented by Chemical Formulae 2 to 7 and Chemical Formulae 24 to 39 according to exemplary embodiments of the present invention is illustrated as follows.

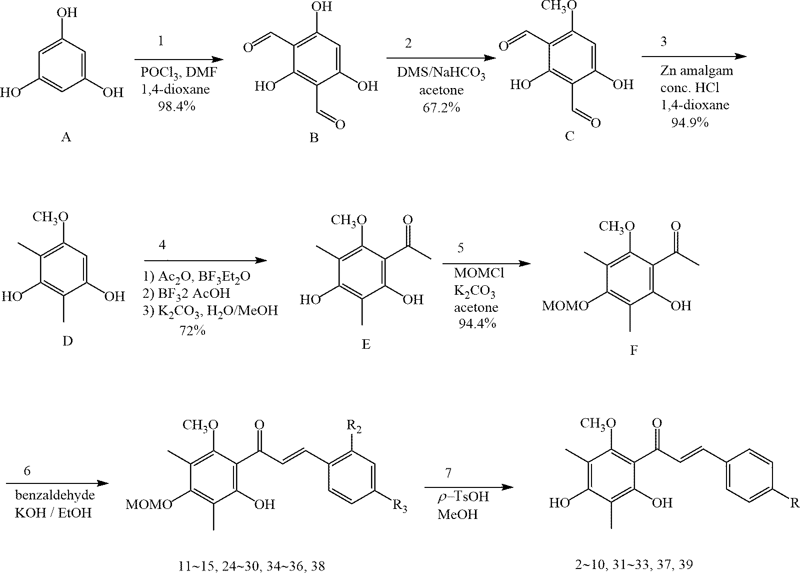

### Reaction Step 1

59.30 mL (6.34 mol) of dimethylformamide was added to a two-necked round-bottom flask, and 49.12 mL (6.34 mol) of phosphorous (V) oxychloride was added dropwise thereto at 0° C. using a dropping funnel, and the resulting mixture was vigorously stirred for 30 minutes. After anhydrous phloroglucinol (40 g, 3.17 mol) of Chemical Formula A was dissolved in 1,4-dioxane (200 mL), the resulting solution was vigorously stirred while being added dropwise to a Vilsmeyer reagent previously prepared at 0° C. The solution was stirred at room temperature for 4 hours and a yellow solid was obtained. This compound was transferred to a 2-L round-bottom flask, DI water (1.5 L) was added thereto, and the resulting mixture was vigorously stirred for 3 hours. After stirring, the precipitated yellow solid was filtered and dried in a vacuum oven at 30° C. for 12 hours to obtain a pale orange compound of Chemical Formula B (56.84 g, 98.4%).

mp = 221-224° C.; TLC R_{f} = 0.208 (n-hexane: acetone = 1:2); IR vmax (cm⁻¹) 2887.88, 1598.70, 1503.24, 1438.64, 1393.32, 1253.50, and 1186.97; ¹H NMR (300 MHz, DMSO-d₆) δ 12.52 (br s, 2H, —OH), 10.01 (s, 2H, CHO), 5.90 (s, 1H, ArH); and ¹³C NMR (150 MHz, DMSO-d₆) δ 191.37 (2C), 169.42 (2C), 169.02 (1C), 103.77 (2C) and 94.07 (1C).

### Reaction Step 2

After the compound of Chemical Formula B (9 g, 49.42 mmol) was put into a round-bottom flask, a nitrogen atmosphere was created and 500 mL of dry acetone was injected using a syringe. After stirring for 10 minutes, dimethyl sulfate (5.16 mL, 54.36 mmol) and sodium bicarbonate (1.66 g, 19.77 mmol) were added thereto. Sodium bicarbonate (1.66 g, 19.77 mmol) was further added twice to the mixture at 12 hour intervals, and the resulting mixture was stirred at 42° C. for 8 days. After the reaction was completed, the mixture was cooled to room temperature and then diluted with ethyl acetate. The organic layer was washed using a 1% aqueous HCl solution, water, and a saturated aqueous NaCl solution, and dried using MgSO₄. The solvent was removed by distillation under reduced pressure and the residue was separated using column chromatography (n-hexane : acetone = 20:1) to obtain a white compound of Chemical Formula C (6.51 g, 67.2%).

mp = 139-140° C.; TLC R_{f} = 0.647 (n-hexane: acetone = 3:2); IR vₘₐₓ (cm⁻¹) 2897.52, 1614.13, 1593.88, 1188.90, and 1080.90; ¹H NMR (300 MHz, CDCl₃) δ 13.64 (s, 1H, —OH), 13.09 (s, 1H, —OH), 10.18 (s, 1H, —CHO), 10.05 (s, 1H, —CHO), 5.92 (s, 1H, Ar—H), 3.95 (s, 3H, —OCH₃); and ¹³C NMR (150 MHz, DMSO-d₆) δ 191.85 (1C), 191.48 (1C), 171.21 (1C), 168.89 (1C), 168.87 (1C), 104.60 (1C), 104.53 (1C) 91.96 (1C), and 57.46 (1C).

### Reaction Step 3

After zinc powder (30 g) was stirred in a 1% HCl aqueous solution (300 mL) for 1 hour, mercury (II) chloride (0.9 g) and a 3% HCl aqueous solution (150 mL) were added thereto and the resulting mixture was vigorously stirred at room temperature for 4 hours to prepare a zinc amalgam. After the amalgam was filtered and washed with 1,4-dioxane, it was put into a solution of the compound of Chemical Formula C and 1,4-dioxane (200 mL). A 36% HCl aqueous solution (12 mL) was slowly added to the mixture at 0° C. and the resulting mixture was stirred for 30 minutes. The reaction mixture was filtered and diluted with ethyl acetate. The organic layer was washed with water and a saturated NaCl aqueous solution and dried using MgSO₄. Thereafter, the solvent was removed by distillation under reduced pressure. The product was separated using column chromatography (n-hexane : acetone = 30 : 1) to obtain a white compound of Chemical Formula D (2.44 g, 94.9%).

mp = 95-96° C.; TLC R_{f} = 0.500 (n-hexane: acetone = 3:2); IR vₘₐₓ (cm⁻¹) 3375.78, 2920.66, 2852.20, 1615.09, 1505.17, 1454.06, 1329.68, 1275.68, 1208.18, 1112.73, and 1088.62; ¹H NMR (300 MHz, DMSO-d₆) δ 8.83 (s, 1H, — OH), 7.95 (s, 1H, —OH), 6.01 (s, 1H, Ar—H), 3.63 (s, 3H, —OCH₃), 1.91(s, 3H, —CH₃) and 1.90 (s, 3H, —CH₃); and ¹³C NMR (150 MHz, DMSO-d₆) δ 155.92 (1C), 154.41 (1C), 153.99 (1C), 103.70 (1C), 103.15 (1C), 91.50 (1C), 55.48 (1C) 9.21 (1C), and 9.01 (1C).

### Reaction Step 4

After the compound of Chemical FormulaD (5.5 g, 32.7 mmol) was put into a round-bottom flask, acetic anhydride (30.93 mL, 327.2 mmol) was added thereto using a syringe, and boron trifluoride diethyl etherate (4.93 mL, 39.3 mmol) was slowly added thereto at 0° C. The mixture was stirred at room temperature for 1 hour and diluted with ethyl acetate. After the organic layer was washed using a 1% HCl aqueous solution, water and a saturated NaCl aqueous solution and dried using MgSO₄, the solvent was removed by distillation under reduced pressure to obtain a compound of Chemical Formula D-1.

After the compound of Chemical Formula D-1 (7 g, 27.7 mmol) was put into a round-bottom flask, a boron trifluoride-acetic acid complex (15.43 mL, 111.1 mmol) was added thereto using a syringe, and the resulting mixture was refluxed for 4 hours. The mixture was cooled to room temperature, and then diluted with ethyl acetate, the organic layer was washed using a 1% HCl aqueous solution, water, and a saturated NaCl aqueous solution, and dried using MgSO₄, and then the solvent was removed by distillation under reduced pressure to obtain a compound of Chemical Formula D-2.

The compound of Chemical Formula D-2 (4.54 g, 18.0 mmol), methanol/water (1:1 v/v, 30 mL), and potassium carbonate (9.96 g, 72.0 mmol) were added to a round-bottom flask, and the resulting mixture was stirred overnight. The mixture was diluted with ethyl acetate, the organic layer was washed using a 1% HCl aqueous solution, water, and a saturated NaCl aqueous solution, and dried using MgSO₄, and then the solvent was removed by distillation under reduced pressure. The product was separated using column chromatography (n-hexane : acetone = 250 : 1) to obtain a compound of Chemical Formula E as a pale yellow solid. The total yield of the three-step reaction to prepare the compound of Chemical Formula E from Chemical Formula D was 72%.

mp = 156° C.; TLC R_{f} = 0.634 (n-hexane: acetone = 1:1); IR vmax (cm⁻¹) 3268.75, 2920.66, 2852.20, 1604.48, 1573.63, 1438.64, 1419.35, 1366.32, 1305.57, 1271.82, 1214.93, 1189.86, and 1101.15; ¹H NMR (300 MHz, DMSO-d₆) δ 13.63 (s, 1H, —OH), 9.56 (s, 1H, —OH), 3.67 (s, 3H, —OCH₃), 2.62 (s, 3H, —COCH₃), 2.04 (s, 3H, —CH₃), and 1.98 (s, 3H, —CH₃); and ¹³C NMR (150 MHz, DMSO-d₆) δ 203.05 (1C), 161.02 (1C), 160.77 (1C), 158.78 (1C) 109.61 (1C), 107.74 (1C), 106.80 (1C), 61.37 (1C), 31.03 (1C), 9.23 (1C), and 8.20 (1C).

### Reaction Step 5

After the compound of Chemical Formula E (0.95 g, 4.52 mmol) and potassium carbonate (0.75 g, 5.42 mmol) were put into a round-bottom flask, dry acetone (40 mL) and chloromethyl methyl ether (0.42 mL, 5.42 mmol) were added thereto, and the resulting mixture was refluxed for 1 hour. The mixture was cooled to room temperature, and then diluted with ethyl acetate, the organic layer was washed using a 1% HCl aqueous solution, water, and a saturated NaCl aqueous solution, and dried using MgSO₄, and then the solvent was removed by distillation under reduced pressure. The product was separated using column chromatography (n-hexane : acetone = 50 : 1) to obtain a pale yellow compound of Chemical Formula F (1.085 g, 94.4 %).

mp = 59-60° C.; TLC R_{f} = 0.676 (n-hexane: acetone = 1:1); IR vₘₐₓ (cm⁻¹) 2953.45, 2921.63, 2852.20, 1601.55, 1454.06, 1409.71, 1355.71, 1317.14, 1267.97, 1218.79, and 1172.51; ¹H NMR (300 MHz, DMSO-d₆) δ 12.80 (br s, 1H, —OH), 5.00 (s, 3H, —CH₂—O), 3.71 (s, 3H, —OCH₃), 3.51 (s, 3H, —OCH₃), 2.65 (s, 3H, —COCH₃), 2.10 (s, 3H, —CH₃), and 2.04 (s, 3H, —CH₃); and ¹³C NMR (150 MHz, DMSO-d₆) δ 204.33 (1C), 160.79 (1C), 159.09 (1C), 158.21 (1C), 115.34 (1C), 114.69 (1C), 112.42 (1C), 98.96 (1C), 61.52 (1C), 57.14 (1C), 31.59 (1C), 9.76 (1C), and 9.25 (1C).

### Reaction Step 6

The compound of Chemical Formula F (1.42 g, 5.58 mmol) and potassium hydroxide (0.94 g, 16.7 mmol) were put into a round-bottom flask, and then completely dissolved by adding ethanol (30 mL) thereto. Appropriately substituted benzaldehyde (6.70 mmol) was added to this solution, and the resulting mixture was stirred at room temperature for 7 days. After the mixture was diluted with ethyl acetate, the organic layer was washed using a 1% NH₄Cl aqueous solution, water and a saturated NaCl aqueous solution, and dried using MgSO₄, and then the solvent was removed by distillation under reduced pressure. The residue was separated using column chromatography (n-hexane : acetone = 500 : 1) and recrystallized using methanol to obtain compounds of Chemical Formulae 11 to 15, Chemical Formulae 24 to 30, Chemical Formulae 34 to 36, and Chemical Formula 38 as yellow solids.

[Compound of Chemical Formula 11 (1-(2′-hydroxy-4′-(methoxymethoxy)-6′-methoxy-3′,5′-dimethylphenyl)-3-(3,4-difluorophenyl)-2-propen-1-one)] Yield: 84.6%

TLC R_{f} = 0.65 (n-hexane/acetone = 3:2); IR vₘₐₓ (cm⁻¹) 2926, 23611, 1603, 1410, 1111; ¹H NMR (CDCl₃, 600 MHz) δ 12.95 (s, 1H, OH), 7.86 (d, J = 15.64 Hz, 1H, —C═C—H), 7.74 (d, J= 15.64 Hz, 1H, —C═C—H), 7.46 (ddd, J= 11.02 Hz, J= 7.63 Hz, J = 2.00 Hz, 1H, Ar—H), 7.37-7.35 (m, 1H, Ar—H), 7.21 (ddd, J= 9.92 Hz, J = 8.27 Hz, J = 8.21 Hz, 1H, Ar—H), 5.02 (s, 2H, —CH₂), 3.65 (s, 3H, —OCH₃), 3.63 (s, 3H, —OCH₃), 2.19 (s, 3H, —CH₃), 2.17 (s, 3H, —CH₃)

[Compound of Chemical Formula 12 (1-(2′-hydroxy-4′-(methoxymethoxy)-6′-methoxy-3′,5′-dimethyphenyl)-3-(4-chlorophenyl)-2-propen-1-one)] Yield: 86.2%

TLC R_{f} = 0.67 (n-hexane/acetone = 3:2); ¹H NMR (CDCl₃, 600 MHz) δ 13.01 (s, 1H, OH), 7.92 (d, J= 15.69 Hz, 1H, —C═C—H), 7.80 (d, J= 15.69 Hz, 1H, —C═C—H), 7.58 (d, J = 8.50 Hz, 2H, Ar—H), 7.39 (d, J = 8.50 Hz, 2H, Ar—H), 5.02 (s, 2H, —CH₂), 3.65 (s, 3H, —OCH₃), 3.63 (s, 3H, —OCH₃), 2.19 (s, 3H, —OCH₃), 2.17 (s, 3H, —OCH₃); ¹³C NMR (CDCl₃, 150 MHz) δ 193.9 (1C), 161.8 (1C), 161.6 (1C), 158.6 (1C), 142.0 (1C), 136.4 (1C), 133.8 (1C), 129.7 (2C), 129.4 (2C), 127.2 (1C), 116.1 (1C), 116.0 (1C), 112.1 (1C), 99.5 (1C), 62.5 (1C), 57.9 (1C), 9.7 (1C), 9.6 (1C).

[Compound of Chemical Formula 13 (1-(2′-hydroxy-4′-(methoxymethoxy)-6′-methoxy-3′,5′-dimethyphenyl)-3-(4-methylthiophenyl)-2-propen-1-one)] Yield: 85.9%

TLC R_{f} = 0.59 (n-hexane/acetone = 3:2); ¹H NMR (CDCl₃, 600 MHz) δ 13.13 (s, 1H, OH), 7.92 (d, J = 15.93 Hz, 1H, —C═C—H), 7.82 (d, J = 15.93 Hz, 2H, —C═C—H), 7.56 (d, J = 6.84 Hz, 1H, Ar—H), 7.25 (d, J = 6.84 Hz, 2H, Ar—H), 5.02 (s, 2H, —CH₂), 3.65 (s, 3H, —OCH₃), 3.63 (s, 3H, —OCH₃), 2.52 (s, 3H, —SCH₃), 2.19 (s, 3H, —CH₃), 2.17 (s, 3H, —CH₃); ¹³C NMR (CDCl₃, 150 MHz) δ 194.0 (1C), 161.6 (1C), 161.5 (1C), 158.6 (1C), 143.2 (1C), 142.4 (1C), 131.8 (1C), 129.0 (2C), 126.2 (1C), 125.6 (1C), 116.0 (1C), 115.9 (1C), 112.1 (1C), 99.4 (1C), 62.4 (1C), 57.8 (1C), 15.3 (1C), 9.7 (1C), 9.6 (1C).

[Compound of Chemical Formula 14 (1-(2′-hydroxy-4′-(methoxymethoxy)-6′-methoxy-3′,5′-dimethylphenyl)-3-(4-(trifluoromethyl)phenyl)-2-propen-1-one)] Yield: 88.4%

¹H NMR (CDCl₃, 600 MHz) δ 12.95 (s, 1H, OH), 8.01 (d, J= 15.71 Hz, 1H, —C═C—H), 7.83 (d, J = 15.71 Hz, 1H, —C═C—H), 7.74 (d, J = 8.06 Hz, 2H, Ar—H), 7.67 (d, J = 8.06 Hz, 2H, Ar—H), 5.03 (s, 2H, —CH₂), 3.66 (s, 3H, —OCH₃), 3.63 (s, 3H, —OCH₃), 2.20 (s, 3H, —CH₃), 2.18 (s, 3H, —CH₃); ¹³C NMR (CDCl₃, 150 MHz) δ 193.8 (1C), 162.0 (1C), 161.7 (1C), 158.7 (1C), 141.1 (1C), 138.7 (1C), 131.7 (d, J= 291.74 Hz, 1C), 129.1 (q, J = 35.05 Hz, 1C), 129.1 (1C), 128.6 (2C), 126.0 (q, J= 3.82 Hz, 2C), 116.2 (1C), 116.1 (1C), 112.0 (1C), 99.5 (1C), 66.3 (1C), 62.5 (1C), 9.7 (1C), 9.6 (1C).

[Compound of Chemical Formula 15 (1-(2′-hydroxy-4′-(methoxymethoxy)-6′-methoxy-3′,5′-dimethylphenyl)-3-(2,5-dimethoxyphenyl)-2-propen-1-one)] Yield: 81.8%

TLC R_{f} = 0.60 (n-hexane/acetone = 3:2); IR vₘₐₓ (cm⁻¹) 2935, 2358, 1624, 1457, 1143; ¹H NMR (CDCl₃, 300 MHz) δ 13.21 (s, 1H, OH), 8.21 (d, J = 15.80 Hz, 1H, —C═C—H), 7.98 (d, J= 15.80 Hz, 1H, —C═C—H), 7.20 (d, J= 2.96 Hz, 1H, Ar—H), 6.94 (dd, J = 8.82 Hz, J = 2.96 Hz, 1H, Ar—H), 6.88 (d, J = 8.82 Hz, 1H, Ar—H), 5.02 (s, 2H, —CH₂), 3.88 (s, 3H, —OCH₃), 3.82 (s, 3H, —OCH₃), 3.66 (s, 3H, —OCH₃), 3.63 (s, 3H, —OCH₃), 2.19 (s, 3H, —CH₃), 2.17 (s, 3H, —CH₃); ¹³C NMR (DMSO-d₆, 150 MHz) δ 194.5 (1C), 161.6 (1C), 161.4 (1C), 158.7 (1C), 153.7 (1C), 153.5 (1C), 143.2 (1C), 138.7 (1C), 127.1 (1C), 117.2 (1C), 115.9 (1C), 115.8 (1C), 113.5 (1C), 112.6 (1C), 112.2 (1C), 99.4 (1C), 62.4 (1C), 57.8 (1C), 56.3 (1C), 56.0 (1C), 9.7 (1C), 9.6 (1C).

[Compound of Chemical Formula 24 (1-(2′-hydroxy-4′-(methoxymethoxy)-6′-methoxy-3′,5′-dimethylphenyl)-3-(4-fluorophenyl)-2-propen-1-one)] Yield: 91.7%

mp = 68° C.; TLC R_{f} = 0.719 (n-hexane: acetone = 1:1); IR vₘₐₓ (cm⁻¹) 3440.94 2943.39, 1628.88, 1603.67, 1508.86, 1143.68, and 1113.05; ¹H NMR (600 MHz, CDCl₃) δ 13.03 (s, 1H, OH), 7.907.87 (d, J = 15.65 Hz, 1H, C═CH), 7.847.81 (d, J = 15.62 Hz, 1H, C═CH), 7.657.62 (q, J = 8.28 Hz, J = 5.53 Hz, 2H, ArH), 7.127.09 (t, J = 8.46 Hz, J = 8.46 Hz, 2H, ArH), 5.02 (2H, —CH2—O—), 3.66 (s, 3H, OCH₃), 3.62 (s, 3H, OCH₃), 2.19 (s, 3H, CH₃), and 2.17 (s, 3H, CH₃); and ¹³C NMR (151 MHz, DMSO-d₆) δ 194.14(1C), 164.69163.04(1C), 160.54(1C), 158.49(1C), 157.63(1C), 142.76(1C), 131.69131.67(1C), 131.39131.33 (2C), 127.18127.17 (1C), 116.64116.50(2C), 116.17(1C), 115.43(1C), 114.09(1C), 99.45(1C), 62.42(1C), 57.60 (1C), 9.98(1C), and 9.92(1C).

[Compound of Chemical Formula 25 (1-(2′-hydroxy-4′-(methoxymethoxy)-6′-methoxy-3′,5′-dimethylphenyl)-3-phenyl-2-propen-1-one)] Yield: 93.1%

mp = 66° C.; TLC R_{f} = 0.703 (n-hexane: acetone = 1:1); IR vₘₐₓ (cm⁻¹) 3461.57, 2958.27, 1630.64, 1563.19, 1345.28, 1144.75, 1113.27, 1057.07, 940.13 and 1225.54; ¹H NMR (600 MHz, CDCl₃) δ 13.08 (s, 1H, OH), 7.987.95 (d, J = 15.65 Hz, 1H, C═CH), 7.887.86 (d, J = 15.68 Hz, 1H, C═CH), 7.667.65 (m, 2H, ArH), 7.447.39 (m, 3H, ArH), 5.02 (s, 2H, —CH2—O—), 3.67 (s, 3H, OCH₃), 3.63 (s, 3H, OCH₃), 2.20 (s, 3H, CH₃), and 2.18 (s, 3H, CH₃); and ¹³C NMR (151 MHz, DMSO-d₆) δ 194.23 (1C), 160.54 (1C), 158.58 (1C), 157.65 (1C), 143.96 (1C), 135.01 (1C), 131.13 (1C), 129.55(2C), 129.00(2C), 127.30 (1C), 116.12 (1C), 115.44 (1C), 114.10 (1C), 99.46 (1C), 62.43 (1C), 57.61 (1C), 10.00 (1C), and 9.93 (1C).

[Compound of Chemical Formula 26 (1-(2′-hydroxy-4′-(methoxymethoxy)-6′-methoxy-3′,5′-dimethylphenyl)-3-(4-tolyl)-2-propen-1-one)] Yield: 85.2%

mp = 61° C.; TLC R_{f} = 0.721 (n-hexane: acetone = 1:1); IR vₘₐₓ (cm⁻¹) 3391.10, 2932.35, 1625.94, 1601.35, 1586.41, 1547.84, 1341.49, 1163.11, 1137.07, 1096.09, 1060.42 and 980.87; ¹H NMR (600 MHz, CDCl₃) δ 13.12 (s, 1H, —OH), 7.94-7.92 (d, J = 15.61 Hz, 1H, —C═C—H), 7.87-7.85 (d, J = 15.64 Hz, 1H, —C═C—H), 7.56-7.55 (d, J = 7.81 Hz, 2H, Ar—H), 7.23-7.22 (d, J = 7.82 Hz, 2H, Ar—H), 5.02 (s, 2H, —CH2—O—), 3.66 (s, 3H, —OCH₃), 3.62 (s, 3H, —OCH₃), 2.40 (s, 3H, —CH₃), 2.20 (s, 3H, —CH₃), and 2.17 (s, 3H, —CH₃); and ¹³C NMR (151 MHz, DMSO-d₆) δ 194.18 (1C), 160.49 (1C), 158.63 (1C), 157.64 (1C), 144.22 (1C), 141.30 (1C), 132.29 (1C), 130.18(2C), 129.05(2C), 126.20 (1C), 116.11 (1C), 115.41 (1C), 114.02 (1C), 99.45 (1C), 62.40 (1C), 57.60 (1C), 21.52 (1C), 9.99 (1C), and 9.92 (1C).

[Compound of Chemical Formula 27 (1-(2′-hydroxy-4′-(methoxymethoxy)-6′-methoxy-3′,5′-dimethylphenyl)-3-(4-isopropylphenyl)-2-propen-1-one)] Yield: 83.9%

mp = 65° C.; TLC R_{f} =0.730 (n-hexane: acetone = 1:1); IR vₘₐₓ (cm⁻¹) 3420.22, 2961.13, 1633.54, 1560.36, 1344.72, 1141.05, 1110.29, and 985.97; ¹H NMR (600 MHz, CDCl₃) δ 13.12 (s, 1H, —OH), 7.95-7.92 (d, J = 15.65 Hz, 1H, —C═C—H), 7.88-7.86 (d, J = 15.57 Hz, 1H, —C═C—H), 7.60-7.58 (d, J = 7.9 Hz, 2H, Ar—H), 7.29-7.27 (d, J = 7.94 Hz, 2H, Ar—H), 5.02 (s, 2H, —CH2—O—), 3.66 (s, 3H, —OCH₃), 3.63 (s, 3H, —OCH₃), 2.98-2.91 (septet, J = 6.96 Hz, 1H, —CH), 2.20 (s, 3H, —CH₃), 2.17 (s, 3H, —CH₃), and 1.28-1.27 (d, J = 6.91 Hz, 6H, —CH₃); and ¹³C NMR (151 MHz, DMSO-d₆) δ 194.16 (1C), 160.56 (1C), 158.74 (1C), 157.70 (1C), 152.03 (1C), 144.18 (1C), 132.70 (1C), 129.19(2C), 127.55(2C), 126.23 (1C), 116.12 (1C), 115.41 (1C), 113.93 (1C), 99.45 (1C), 62.42 (1C), 57.60 (1C), 33.86 (1C), 24.03(2C), 9.99 (1C), and 9.92 (1C).

[Compound of Chemical Formula 28 (1-(2′-hydroxy-4′-(methoxymethoxy)-6′-methoxy-3′,5′-dimethylphenyl)-3-(4-methoxymethoxyphenyl)-2-propen-1-one)] Yield: 83.1%

mp = 66° C.; TLC R_{f}= 0.633 (n-hexane: acetone = 1:1); IR vₘₐₓ (cm⁻¹) 3431.67, 2958.57, 1631.02, 1152.50, 1140.45, 1057.15, 1054.54, 1005.70//1632.64, 1560.69, 1512.01, and 1156.51; ¹H NMR (600 MHz, CDCl₃) δ 13.14 (s, 1H, —OH), 7.88-7.86 (d, J = 15.61 Hz, 1H, —C═C—H), 7.86-7.83 (d, J = 15.64 Hz, 1H, —C═C—H), 7.61-7.59 (d, J = 7.81 Hz, 2H, Ar—H), 7.08-7.06 (d, J = 7.82 Hz, 2H, Ar—H), 5.22 (s, 2H, —CH2—O—), 5.02 (s, 2H, —CH2—O—), 3.66 (s, 3H, —OCH₃), 3.62 (s, 3H, —OCH₃), 3.49 (s, 3H, —OCH₃), 2.19 (s, 3H, —CH₃), and 2.17 (s, 3H, —CH₃); and ¹³C NMR (151 MHz, DMSO-d₆) δ 194.04 (1C), 160.41 (1C), 159.32 (1C), 158.69 (1C), 157.62 (1C), 144.07 (1C), 130.84(2C), 128.57 (1C), 125.16 (1C), 116.99(2C), 116.03 (1C), 115.38 (1C), 114.01 (1C), 99.44 (1C), 94.15 (1C), 62.38 (1C), 57.60 (1C), 56.20 (1C), 10.00 (1C), and 9.93 (1C).

[Compound of Chemical Formula 29 (1-(2′-hydroxy-4′-(methoxymethoxy)-6′-methoxy-3′,5′-dimethylphenyl)-3-(4-methoxyphenyl)-2-propen-1-one)] Yield: 92.7%

mp = 88° C.; TLC R_{f}= 0.650 (n-hexane: acetone = 1:1); IR vₘₐₓ (cm⁻¹) 3530.27 2945.69, 1625.57, 1548.34, 1511.09, 1172.30, 1141.29 1113.79, and 939.40; ¹H NMR (600 MHz, CDCl₃) δ 13.16 (s, 1H, —OH), 7.89-7.86 (d, J = 15.76 Hz, 1H, —C═C—H), 7.86-7.83 (d, J = 15.68 Hz, 1H, —C═C—H), 7.62-7.60 (d, J = 8.77 Hz, 2H, Ar—H), 6.94-6.93 (d, J = 8.76 Hz, 2H, Ar—H), 5.02 (s, 2H, —CH2—O—), 3.86 (s, 3H, —OCH₃), 3.66 (s, 3H, —OCH₃), 3.62 (s, 3H, —OCH₃), 2.19 (s, 3H, —CH₃), and 2.17 (s, 3H, —CH₃); and ¹³C NMR (151 MHz, DMSO-d₆) δ194.01 (1C), 161.94 (1C), 160.37 (1C), 158.67 (1C), 157.61 (1C), 144.37 (1C), 130.96(2C), 127.60 (1C), 124.63 (1C), 116.05 (1C), 115.37 (1C), 115.08(2C), 113.99 (1C), 99.44 (1C), 62.37 (1C), 57.60 (1C), 55.86 (1C), 9.99 (1C), and 9.92 (1C).

[Compound of Chemical Formula 30 (1-(2′-hydroxy-4′-(methoxymethoxy)-6′-methoxy-3′,5′-dimethylphenyl)-3-(4-(N,N-dimethylamino)phenyl)-2-propen-1-one)] Yield: 76.4%

TLC R_{f} = 0.57 (n-hexane/acetone = 3:2); ¹H NMR (DMSO-d₆, 600 MHz) δ12.64 (s, 1H, OH), 7.73 (d, J=15.46 Hz, 1H, —C═C—H), 7.56 (d, J=8.94 Hz, 2H, Ar—H), 7.54 (d, J=15.46 Hz, 1H, —C═C—H), 6.74 (d, J=7.48 Hz, 2H, Ar—H), 5.0(s, 2H, —CH₂), 3.62 (s, 3H, —OCH₃), 3.52 (s, 3H, —OCH₃), 3.01 (s, 6H, —N—(CH₃)₂), 2.11 (s, 3H, —CH₃), 2.06 (s, 3H, —CH₃); ¹³C NMR (DMSO-d₆, 600 MHz) δ 192.9 (1C), 159.7 (1C), 158.7 (1C), 157.1 (1C), 152.1 (1C), 145.6 (1C), 130.6 (2C), 121.7 (1C), 120.2 (1C), 115.4 (1C), 114.7 (1C), 113.1 (1C), 111.9 (2C), 98.97 (1C), 61.8 (1C), 57.1 (1C), 39.6 (2C), 9.5 (1C), 9.4 (1C)

[Compound of Chemical Formula 34 (1-(2′-hydroxy-4′-(methoxymethoxy)-6′-methoxy-3′,5′-dimethylphenyl)-3-(2-fluorophenyl)-2-propen-1-one)] Yield: 86.2%

¹H NMR (CDC13, 600 MHz) δ13.08 (s, 1H, OH), 8.04 (d, J=15.83 Hz, 1H, —C═C—H), 7.98 (d, J=15.83 Hz, 1H, —C═C—H), 7.67 (dt, J=7.51 Hz, J=1.69 Hz, 1H, Ar—H), 7.37 (dq, J=8.04 Hz, J=7.41 Hz, J=1.69 Hz, 1H, Ar—H), 7.19 (t, J=7.41 Hz, 1H, Ar—H), 7.13 (dd, J=10.40 Hz, J=8.04 Hz, 1H, Ar—H), 5.02 (s, 2H, —CH₂), 3.67 (s, 3H, —OCH₃), 3.62 (s, 3H, —OCH₃), 2.19 (s, 3H, —OCH₃), 2.17 (s, 3H, —OCH₃); ¹³C NMR (CDCl₃, 150 MHz) δ 194.1 (1C), 161.9 (d, J=254.55 Hz, 1C), 161.8 (1C), 161.7 (1C), 158.7 (1C), 135.9 (d, J=2.68 Hz, 1C), 131.8 (d, J=8.81 Hz, 1C), 129.4 (d, J=2.88 Hz, 1C), 129.1 (d, J=6.47 Hz, 1C), 124.6 (d, J=3.62 Hz, 1C), 123.5 (1C), 123.4 (1C), 116.4 (d, J=21.98 Hz, 1C), 116.1 (d, J=23.04 Hz, 1C), 112.1 (1C), 99.4 (1C), 62.5 (1C), 57.9 (1C), 9.8 (1C), 9.6 (1C)

[Compound of Chemical Formula 35 (1-(2′-hydroxy-4′-(methoxymethoxy)-6′-methoxy-3′,5′-dimethylphenyl)-3-(3-fluorophenyl)-2-propen-1-one)] Yield: 88.5%

TLC R_{f} = 0.59 (n-hexane/acetone = 3:2); ¹H NMR (DMSO-d₆, 600 MHz) δ11.83z (s, 1H, OH), 7.70 (d, J=15.82 Hz, 1H, —C═C—H), 7.65 (d, J=15.82 Hz, 1H, —C═C—H), 7.62 (dd, J=10.15 Hz, J=2.33 Hz 1H, Ar—H), 7.59 (d, J=7.78 Hz, 1H, Ar—H), 7.49 (ddd, J=8.15 Hz, J=7.78 Hz, J=5.98 Hz, 1H, Ar—H), 7.28 (ddd, J=10.89 Hz, J=8.15 Hz, J=2.33 Hz, 1H, Ar—H), 5.02(s, 2H, —CH₂), 3.61 (s, 3H, —OCH₃), 3.52 (s, 3H, —OCH₃), 2.11 (s, 3H, —CH₃), 2.08 (s, 3H, —CH₃); ¹³C NMR (DMSO-d₆, 150 MHz) δ 193.7 (1C), 162.4 (d, J = 244.14 Hz, 1C), 160.1 (1C), 157.8 (1C), 157.1 (1C), 141.7 (1C), 137.5 (d, J = 8 Hz, 1C), 131.0 (d, J = 8.4 Hz, 1C), 128.4 (1C), 124.7 (d, J=2.54 Hz, 1C), 117.2 (d, J=21.35 Hz, 1C), 115.7 (1C), 115.0 (1C), 114.8 (d, J = 21.93 Hz, 1C), 113.8 (1C), 99.0 (1C), 61.9 (1C), 57.1 (1C), 9.5 (1C), 9.4 (1C).

[Compound of Chemical Formula 36 (1-(2′-hydroxy-4′-(methoxymethoxy)-6′-methoxy-3′,5′-dimethylphenyl)-3-(4-ethoxy-3-fluorophenyl)-2-propen-1-one)] Yield: 75.2%

mp. 80-81° C.; TLC R_{f}= 0.62 (n-hexane/acetone = 3:2); IR vₘₐₓ (cm⁻¹) 2935, 2359, 1627, 1425, 1146; ¹H NMR (CDCl₃, 600 MHz) δ 13.08 (s, 1H, OH), 7.82 (d, J = 15.63 Hz, 1H, —C═C—H), 7.78 (d, J = 15.63 Hz, 1H, —C═C—H), 7.41 (dd, J = 12.00 Hz, J = 2.47 Hz, 1H, Ar—H), 7.33 (d, J = 8.55 Hz, 1H, Ar—H), 6.96 (t, J = 8.55 Hz, 1H, Ar—H), 5.02 (s, 2H, —CH₂), 4.16 (q, J = 6.99 Hz, 2H, —CH₂), 3.66 (s, 3H, —OCH₃), 3.62 (s, 3H, —OCH₃), 2.19 (s, 3H, —CH₃), 2.17 (s, 3H, —CH₃), 1.48 (t, J = 6.99 Hz, 3H, —CH₃); ¹³C NMR (DMSO-d₆, 150 MHz) δ 193.8 (1C), 161.6 (1C), 161.6 (1C), 158.9 (1C), 158.6 (1C), 152.8 (d, J = 246.81 Hz, 1C), 149.2 (d, J = 10.95 Hz, 1C), 142.5 (d, J = 2.51 Hz, 1C), 128.5 (d, J = 6.46 Hz, 1C), 126.1 (d, J = 3.15 Hz, 1C), 125.4 (1C), 116.0 (1C), 115.9 (1C), 115.1 (d, J= 18.75 Hz, 1C), 114.4 (1C), 112.1 (1C), 99.4 (1C), 65.0 (1C), 62.4 (1C), 57.8 (1C), 14.8 (1C), 9.7 (1C), 9.6 (1C).

[Compound of Chemical Formula 38 (1-(2′-hydroxy-4′-(methoxymethoxy)-6′-methoxy-3′,5′-dimethylphenyl)-3-(2-chlorophenyl)-2-propen-1-one)] Yield: 85.7%

¹H NMR (CDCl₃, 600 MHz) 812.98 (s, 1H, OH), 8.24 (d, J= 15.66 Hz, 1H, —C═C—H), 7.92 (d, J=15.66 Hz, 1H, —C═C—H), 7.76 (dd, J=7.31 Hz, J=2.17 Hz, 1H, Ar—H), 7.45 (dd, J=7.56 Hz, J=1.76 Hz, 1H, Ar—H), 7.34-7.29 (m, 2H, Ar—H), 5.02 (s, 2H, —CH₂), 3.66 (s, 3H, —OCH₃), 3.62 (s, 3H, —OCH₃), 2.19 (s, 3H, —OCH₃), 2.17 (s, 3H, —OCH₃); ¹³C NMR (CDCl₃, 150 MHz) δ 193.8 (1C), 161.7 (1C), 161.5 (1C), 158.5 (1C), 138.7 (1C), 135.6 (1C), 133.4 (1C), 131.1 (1C), 130.3 (1C), 128.9 (1C), 127.7 (1C), 127.1 (1C), 115.9 (1C), 115.8 (1C), 99.3 (1C), 62.4 (1C), 57.7 (1C), 9.5 (1C), 9.4 (1C).

### Reaction Step 7

After one compound (2.3 mmol) of Chemical Formulae 11 to 15, Chemical Formulae 24 to 30, Chemical Formulae 34 to 36, and Chemical Formula 38 and 30 mL of methanol were put into a round-bottom flask, p-toluenesulfonic acid (0.528 g, 2.76 mmol) was added thereto, and the resulting mixture was stirred at room temperature for 24 hours. After the mixture was diluted with ethyl acetate, the organic layer was washed using a 1% HCl aqueous solution, water and a saturated NaCl aqueous solution, and dried using MgSO₄, and then the solvent was removed by distillation under reduced pressure. The residue was separated using column chromatography (n-hexane : acetone = 500 : 1) to obtain white compounds of Chemical Formulae 2 to 10, Chemical Formulae 31 to 33, Chemical Formula 37, and Chemical Formula 39.

[Compound of Chemical Formula 2 (1-(2′,4′-dihydroxy-6′-methoxy-3′,5′-dimethylphenyl)-3-(4-fluorophenyl)-2-propen-l-one)] Yield: 90.1%

mp = 110° C.; TLC R_{f} = 0.623 (n-hexane: acetone = 1:1); IR vₘₐₓ (cm⁻¹) 3375.95, 2926.49, 1630.57, 1604.34, 1543.85, 1510.49, 1416.08, 1237.34, 1165.80, 1157.02, and 1113.64; ¹H NMR (600 MHz, CDCl₃) δ 13.56 (s, 1H, OH), 7.927.89 (d, J = 15.65 Hz, 1H, C═CH), 7.817.79 (d, J = 15.66 Hz, 1H, C═CH), 7.647.62 (q, J = 8.55 Hz, J = 5.45 Hz, 2H, Ar—H), 7.11-7.09 (t, J = 8.56 Hz, J = 8.56 Hz, 2H, Ar—H), 5.37 (s, 1H, —OH), 3.66 (s, 3H, —OCH₃), 2.15 (s, 3H, —CH₃), and 2.13 (s, 3H, —CH₃); and ¹³C NMR (151 MHz, DMSO-d₆) δ 192.76(1C), 164.53-162.88(1C), 161.70(1C), 161.66(1C), 158.73(1C), 141.65(1C), 132.01-131.99(1C), 131.14-131.08(2C), 126.92-126.91(1C), 116.63-116.46(2C), 110.59(1C), 108.25(1C), 107.60(1C), 62.34(1C), 9.40(1C), and 8.78(1C).

[Compound of Chemical Formula 3 (1-(2′,4′-dihydroxy-6′-methoxy-3′,5′-dimethylphenyl)-3-(4-tolyl)-2-propen-1-one)] Yield: 90.5%

mp = 126° C.; TLC R_{f} = 0.656 (n-hexane: acetone = 1:1); IR vₘₐₓ (cm⁻¹) 3373.74, 2917.05, 1625.81, 1609.97 1540.24, 1359.81, 1163.45, and 1111.58; ¹H NMR (600 MHz, CDCl₃) δ 13.65 (s, 1H, —OH), 7.96-7.94 (d, J = 15.62 Hz, 1H, —C═C—H), 7.85-7.82 (d, J = 15.67 Hz, 1H, —C═C—H), 7.55-7.54 (d, J = 7.72 Hz, 2H, Ar—H), 7.23-7.21 (d, J = 7.76 Hz, 2H, Ar—H), 5.42 (s, 1H, —OH), 3.66 (s, 3H, —OCH₃), 2.39 (s, 3H, —CH₃), 2.15 (s, 3H, —CH₃), and 2.13 (s, 3H, —CH₃); and ¹³C NMR (151 MHz, DMSO-d₆) δ 192.78(1C), 161.73(1C), 161.65(1C), 158.70(1C), 143.07(1C), 140.95(1C), 132.63(1C), 130.17 (2C), 128.84 (2C), 125.93(1C), 110.57(1C), 108.20(1C), 107.58(1C), 62.30(1C), 21.51(1C), 9.41(1C), and 8.79(1C).

[Compound of Chemical Formula 4 (1-(2′,4′-dihydroxy-6′-methoxy-3′,5′-dimethylphenyl)-3-(4-isopropylphenyl)-2-propen-1-one)] Yield: 98.4%

mp = 101° C.; TLC R_{f} = 0.656 (n-hexane: acetone = 1:1); IR vₘₐₓ (cm⁻¹) 3343.73, 2956.05, 1628.47, 1605.90, 1552.23, 1354.84, 1166.48, and 1113.41; ¹H NMR (600 MHz, CDCl₃) δ 13.65 (s, 1H, —OH), 7.97-7.94 (d, J = 15.65 Hz, 1H, —C═C—H), 7.86-7.83 (d, J = 15.62 Hz, 1H, —C═C—H), 7.59-7.58 (d, J = 7.99 Hz, 2H, Ar—H), 7.28-7.27 (d, J = 7.97 Hz, 2H, Ar—H), 5.36 (s, 1H, —OH), 3.66 (s, 3H, —OCH₃), 2.97-2.92 (septet, J = 6.92 Hz, 1H, —CH), 2.16 (s, 3H, —CH₃), 2.13 (s, 3H, —CH₃) and 1.28-1.27 (d, J = 6.89 Hz, 6H, —CH₃); and ¹³C NMR (151 MHz, DMSO-d₆) δ 192.77 (1C), 161.74 (1C), 161.66 (1C), 158.71 (1C), 151.71 (1C), 143.05 (1C), 133.02 (1C), 128.98(2C), 127.54(2C), 125.99 (1C), 110.58 (1C), 108.21 (1C), 107.58 (1C), 62.31 (1C), 33.85 (1C), 24.06(2C), 9.41 (1C), and 8.79 (1C).

[Compound of Chemical Formula 5 (1-(2′,4′-dihydroxy-6′-methoxy-3′,5′-dimethylphenyl)-3-(4-methoxyphenyl)-2-propen-1-one)] Yield: 94.9%

mp = 164° C.; TLC R_{f} = 0.590 (n-hexane: acetone = 1:1); IR vₘₐₓ (cm⁻¹) 3516.40, 2943.09, 1628.13, 1606.73, 1558.04, 1509.35, 1348.72, 1162.34, and 1109.43; ¹H NMR (600 MHz, CDCl₃) δ 13.69 (s, 1H, —OH), 7.89-7.87 (d, J = 15.55 Hz, 1H, —C═C—H), 7.85-7.82 (d, J = 15.62 Hz, 1H, —C═C—H), 7.61-7.59 (d, J = 8.57 Hz, 2H, Ar—H), 6.94-6.92 (d, J = 8.46 Hz, 2H, Ar—H), 5.44 (s, 1H, —OH), 3.85 (s, 3H, —OCH₃), 3.66 (s, 3H, —OCH₃), 2.15 (s, 3H, —CH₃), and 2.13 (s, 3H, —CH₃); and ¹³C NMR (151 MHz, DMSO-d₆) δ 192.71 (1C), 161.73 (1C), 161.70 (1C), 161.38 (1C), 158.64 (1C), 143.24 (1C), 130.70(2C), 127.94 (1C), 124.34 (1C), 115.07(2C), 110.48 (1C), 108.23 (1C), 107.57 (1C), 62.27 (1C), 55.84 (1C), 9.41 (1C), and 8.79 (1C).

[Compound of Chemical Formula 6 (1-(2′,4′-dihydroxy-6′-methoxy-3′,5′-dimethylphenyl)-3-(3,4-difluorophenyl)-2-propen-1-one)] Yield: 89.7%

TLC R_{f}= 0.59 (n-hexane/acetone = 3:2); IR vₘₐₓ (cm⁻¹) 3373, 2922, 2359, 1603, 1457, 1111; ¹H NMR (CDCl₃, 600 MHz) δ 13.47(s, 1H, OH), 7.88 (d, J = 15.69 Hz, 1H, —C═C—H), 7.71(d, J = 15.69 Hz, 1H, —C═C—H), 7.45 (ddd, J =10.93 Hz, J = 7.57 Hz, J = 1.95 Hz 1H, Ar—H), 7.36-7.34 (m, 1H, Ar—H), 7.20 (ddd, J =10.00 Hz, J = 8.33 Hz, J = 8.20 Hz, Ar—H), 5.38 (s, 1H, OH), 3.64 (s, 3H, —OCH₃), 2.15 (s, 3H, —CH₃), 2.13 (s, 3H, —CH₃); ¹³C NMR (DMSO-d₆, 150 MHz) δ 192.9 (1C), 162.2 (1C), 159.6 (1C), 158.9 (1C), 151.4 (dd, J = 244.34 Hz, 13.03 Hz, 1C), 150.6 (dd, J = 249.48 Hz, J = 13.11 Hz, 1C), 140.4 (1C), 127.9 (1C), 125.3 (d, J = 3.45 Hz, 1C), 125.2 (d, J = 3.45 Hz, 1C), 118.0 (d, J = 17.67 Hz, 1C), 116.5 (d, J = 17.34 Hz, 1C), 109.1 (1C), 109.0 (1C) 106.7 (1C), 62.5 (1C), 8.3 (1C), 7.6 (1C).

[Compound of Chemical Formula 7 (1-(2,4′-dihydroxy-6′-methoxy-3′,5′-dimethylphenyl)-3-(4-(N,N-dimethylamino)phenyl)-2-propen-1-one)] Yield: 88.3%

TLC R_{f} = 0.57 (n-hexane/acetone = 3:2); ¹H NMR (CDCl₃, 600 MHz) δ 13.90 (s, 1H, OH), 7.86 (d, J=15.47 Hz, 1H, —C═C—H), 7.81 (d, J=15.47 Hz, 1H, —C═C—H), 7.55 (d, J=8.36 Hz, 2H, Ar—H), 6.69 (d, J=8.36 Hz, 2H, Ar—H), 3.65 (s, 3H, —OCH₃), 3.03 (s, 6H, —N—(CH₃)₂), 2.15 (s, 3H, —CH₃), 2.12 (s, 3H, —CH₃); ¹³C NMR (CDCl₃, 150 MHz) δ 193.2 (1C), 162.1 (1C), 159.0 (1C), 158.7 (1C), 152.1 (1C), 144.6 (1C), 130.5 (2C), 123.3 (1C), 121.3 (1C), 112.1 (2C), 109.2 (1C), 108.9 (1C), 106.8 (1C), 62.3 (1C), 40.2 (2C), 8.4 (1C), 7.8 (1C).

[Compound of Chemical Formula 8 (1-(2′,4′-dihydroxy-6′-methoxy-3′,5′-dimethylphenyl)-3-(4-chlorophenyl)-2-propen-1-one)] Yield: 91.2%

TLC R_{f} = 0.57 (n-hexane/acetone = 3:2); ¹H NMR (DMSO-d₆, 600 MHz) δ 13.57 (s, 1H, OH), 7.92(d, J = 15.50 Hz, 1H, —C═C—Hz), 7.76 (d, J = 8.37 Hz, Ar—H), 7.74 (d, J = 15.50 Hz, 1H, —C═C—H), 7.52 (d, J = 8.37 Hz, 1H, Ar—H), 3.59 (s, 3H, —OCH₃), 2.06 (s, 3H, —CH₃), 2.01 (s, 3H, —CH₃); ¹³C NMR (DMSO-d₆, 150 MHz) δ 192.2(1C), 161.4 (1C), 161.3 (1C), 161.0 (1C), 160.7 (1C), 158.3 (1C), 140.8 (1C), 134.8 (1C), 133.8 (1C), 130.0 (1C), 129.1 (1C), 127.3 (1C), 110.2 (1C), 107.7 (1C), 107.1 (1C), 61.9 (1C), 8.9 (1C), 8.3 (1C).

[Compound of Chemical Formula 9 (1-(2′,4′-dihydroxy-6′-methoxy-3′,5′-dimethylphenyl)-3-(4-(trifluoromethyl)phenyl)-2-propen-1-one)] Yield: 92.0%

¹H NMR (CDCl₃, 600 MHz) δ 13.46 (s, 1H, OH), 8.03 (d, J = 15.69 Hz, 1H, —C═C—H), 7.81 (d, J = 15.69 Hz, 1H, —C═C—H), 7.74 (d, J = 8.09 Hz, 2H, Ar—H), 7.67 (d, J = 8.09 Hz, 2H, Ar—H), 5.37 (s, 1H, OH), 3.65 (s, 3H, —OCH₃), 2.16 (s, 3H, —CH₃), 2.14 (s, 3H, —CH₃); ¹³C NMR (CDCl₃, 150 MHz) δ 193.0 (1C), 162.3 (1C), 159.6 (1C), 159.0 (1C), 140.6 (1C), 138.9 (1C), 134.6 (d, J = 293.62 Hz, 1C), 129.6 (1C), 129.3 (q, J = 35.05 Hz, 1C), 128.5 (2C), 126.0 (q, J = 3.72 Hz, 2C), 109.1 (1C), 109.1 (1C), 106.7 (1C), 62.5 (1C), 8.3 (1C), 7.6 (1C)

[Compound of Chemical Formula 10 (1-(2′,4′-dihydroxy-6′-methoxy-3′,5′-dimethylphenyl)-3-(2,5-dimethoxyphenyl)-2-propen-1-one)] Yield: 92.5%

TLC R_{f}= 0.50 (n-hexane/acetone = 3:2); IR vₘₐₓ (cm⁻¹) 3440, 2924, 2362, 1616, 1457, 1163; ¹H NMR (CDCl₃, 300 MHz) δ 13.75 (s, 1H, OH), 8.18 (d, J = 15.82 Hz, 1H, —C═C—H), 8.00 (d, J = 15.82 Hz, 1H, —C═C—H), 7.20 (d, J = 2.91 Hz, 1H, Ar—H), 6.93 (dd, J = 8.97 Hz, J = 2.91 Hz, 1H, Ar—H), 6.87 (d, J = 8.97 Hz, 1H, Ar—H), 5.75 (s, 1H, OH), 3.87 (s, 3H, —OCH₃), 3.81 (s, 3H, —OCH₃), 3.65 (s, 3H, —OCH₃), 2.14 (s, 3H, —CH₃), 2.12 (s, 3H, —CH₃); ¹³C NMR (DMSO-d₆, 150 MHz) δ 193.8 (1C), 162.2 (1C), 159.1 (1C), 159.0 (1C), 153.7 (1C), 153.4 (1C), 138.1 (1C), 127.3 (1C), 125.2 (1C), 116.9 (1C), 113.4 (1C), 112.6 (1C), 109.3 (1C), 108.8 (1C), 106.6 (1C), 62.5 (1C), 56.2 (1C), 56.0 (1C), 8.4 (1C), 7.7 (1C).

[Compound of Chemical Formula 31 (1-(2′,4′-dihydroxy-6′-methoxy-3′,5′-dimethylphenyl)-3-(2-fluorophenyl)-2-propen-1-one)] Yield: 93.7%

¹H NMR (DMSO-d₆, 600 MHz) δ13.60 (s, 1H, OH), 8.07 (d, J= 15.84 Hz, 1H, —C═C—H), 7.96 (d, J=15.84 Hz, 1H, —C═C—H), 7.66 (t, J=7.64 Hz, 1H, Ar—H), 7.37 (q, J=6.89 Hz, 1H, Ar—H), 7.18 (t, J=7.55 Hz, 1H, Ar—H), 7.12 (t, J=9.41 Hz, 1H, Ar—H), 5.41 (s, 1H, OH), 3.67 (s, 3H, —OCH₃), 2.15 (s, 3H, —CH₃), 2.13 (s, 3H, —CH₃); ¹³C NMR (CDCl₃, 150 MHz) δ 193.4 (1C), 162.3 (1C), 161.7 (d, J=255.19 Hz, 1C), 159.4 (1C), 159.1 (1C), 135.3 (d, J=2.93 Hz, 1C), 131.6 (d, J=8.71 Hz, 1C), 129.3 (d, J=3 Hz, 1C), 129.2 (d, J=6.38 Hz, 1C), 124.6 (d, J=3.58 Hz, 1C), 123.6 (1C), 123.6 (1C), 116.4 (d, J=21.91 Hz, 1C), 109.1 (d, J=24.62 Hz, 1C), 106.7 (1C), 62.5 (1C), 8.4 (1C), 7.7 (1C).

[Compound of Chemical Formula 32 (1-(2′,4′-dihydroxy-6′-methoxy-3′,5′-dimethylphenyl)-3-(3-fluorophenyl)-2-propen-1-one)] Yield: 95.8%

¹H NMR (CDCl₃, 600 MHz) δ 13.51 (s, 1H, OH), 7.96 (d, J = 15.66 Hz, 1H, —C═C—H), 7.77 (d, J = 15.66 Hz, 1H, —C═C—H), 7.41-7.36 (m, 2H, Ar—H), 7.34 (d, J = 9.67 Hz, 1H, Ar—H), 7.11-7.07 (m, 1H, Ar—H), 5.37 (s, 1H, OH), 3.66 (s, 3H, —OCH₃), 2.16 (s, 3H, —CH₃), 2.13 (s, 3H, —CH₃); ¹³C NMR (CDCl₃, 150 MHz) δ 193.3 (1C), 163.3 (d, J=243.91, 1C), 162.3 (1C), 159.6 (1C), 159.1 (1C), 141.4 (d, J=1.65, 1C), 137.9 (d, J=7.75, 1C), 130.6 (d, J=8.4, 1C), 128.3 (1C), 124.7 (d, J= 2.28, 1C), 117.2 (d, J= 20.68, 1C), 114.6 (d, J=21.84, 1C), 109.2 (1C), 109.1 (1C), 106.8 (1C), 62.6 (1C), 8.4 (1C), 7.7 (1C).

[Compound of Chemical Formula 33 (1-(2′,4′-dihydroxy-6′-methoxy-3′,5′-dimethylphenyl)-3-(4-ethoxy-3-fluorophenyl)-2-propen-1-one)] Yield: 88.6%

TLC R_{f}= 0.56 (n-hexane/acetone = 3:2); IR vₘₐₓ (cm⁻¹) 3438, 2924, 2359, 1615, 1436, 1163; ¹H NMR (CDCl₃, 600 MHz) δ 13.61 (s, 1H, OH), 7.84 (d, J = 15.56 Hz, 1H, —C═C—H), 7.75 (d, J = 15.56 Hz, 1H, —C═C—H), 7.40 (dd, J = 12.11 Hz, J = 2.12 Hz, 1H, Ar—H), 7.32 (d, J = 8.46 Hz, 1H, Ar—H), 6.96 (t, J = 8.46 Hz, 1H, Ar—H), 5.51 (s, 1H, OH), 4.16 (q, J = 7.00 Hz, 2H, —CH₂), 3.69 (s, 3H, —OCH₃), 2.15 (s, 3H, —CH₃), 2.12 (s, 3H, —CH₃), 1.47 (t, J = 7.00 Hz, 3H, —CH₃); ¹³C NMR (DMSO-d₆, 150 MHz) δ 193.1 (1C), 162.1 (1C), 159.3 (1C), 158.9 (1C), 152.8 (d, J = 246.65 Hz, 1C), 149.0 (d, J = 10.96 Hz, 1C), 141.9 (d, J = 2.33 Hz, 1C), 128.7 (d, J = 6.63 Hz, 1C), 126.0 (d, J = 3.21 Hz, 1C), 125.6 (1C), 115.0 (d, J = 18.76 Hz, 1C), 114.9 (1C), 109.1 (1C), 109.0 (1C), 106.7 (1C), 65.0 (1C), 62.4 (1C), 14.8 (1C), 8.3 (1C), 7.7 (1C)

[Compound of Chemical Formula 37 (1-(2′,4′-dihydroxy-6′-methoxy-3′,5′-dimethylphenyl)-3-(2-chlorophenyl)-2-propen-1-one)] Yield: 96.1%

¹H NMR (DMSO-d₆, 600 MHz) δ13.50 (s, 1H, OH), 8.22 (d, J=15.67 Hz, 1H, —C═C—H), 7.95 (d, J=15.67 Hz, 1H, —C═C—H), 7.76 (dd, J=6.89 Hz, J=2.55 Hz, 1H, —C═C—H), 7.44 (dd, J=6.81 Hz, J= 2.28 Hz, 1H, —C═C—H), 7.33-7.28 (m, 2H), 3.65 (s, 3H, —OCH₃), 2.15 (s, 3H, —CH₃), 2.13 (s, 3H, —CH₃); ¹³C NMR (CDCl₃, 150 MHz) δ 193.2 (1C), 162.2 (1C), 159.5 (1C), 159.1 (1C), 138.3 (1C), 135.6 (1C), 133.7 (1C), 130.9 (1C), 130.4 (1C), 129.3 (1C), 127.8 (1C), 127.2 (1C), 109.2 (1C), 109.1 (1C), 106.7 (1C), 62.5 (1C), 8.3 (1C), 7.7 (1C).

[Compound of Chemical Formula 39 (1-(2′,4′-dihydroxy-6′-methoxy-3′,5′-dimethylphenyl)-3-(2,4-dimethylphenyl)-2-propen-1-one)] Yield: 95.5%

¹H NMR (DMSO-d₆, 600 MHz) δ13.67 (s, 1H, OH), 9.64 (broad, 1H, OH), 7.99 (d, J= 15.53 Hz, 1H, —C═C—H), 7.83 (J=15.53 Hz, 1H, —C═C—H), 7.63 (d, J=7.73 Hz, 1H, Ar), 7.11-7.10 (m, 2H, Ar), 3.60 (s, 3H, —OCH₃), 2.41(s, 3H, —CH₃), 2.30(s, 3H, —CH₃), 2.06(s, 3H, —CH₃), 2.02(s, 3H, —CH₃); ¹³C NMR (DMSO-d₆, 150 MHz) δ 192.4(1C), 161.3(1C), 161.1(1C), 158.3(1C), 140.1(1C), 139.8(1C), 137.8(1C), 131.5(1C), 130.7(1C), 127.3(1C), 126.4(1C), 126.3(1C), 110.1(1C), 107.8(1C), 107.1(1C), 61.9(1C), 20.9(1C), 19.3(1C), 8.9(1C), 8.3(1C).

Reaction Scheme 2 for preparing compounds represented by Chemical Formulae 16 to 23 according to exemplary embodiments of the present invention is illustrated as follows.

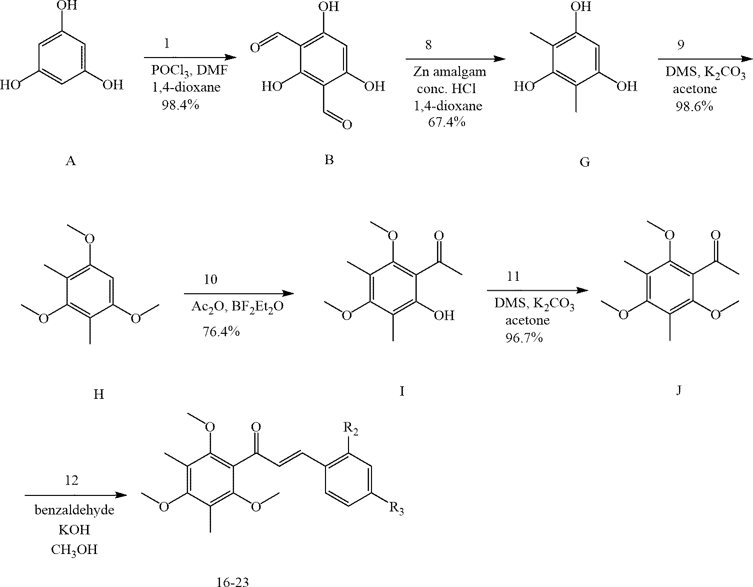

### Reaction Step 1

As described in above <Reaction Step 1>.

### Reaction Step 8

After 300 mL of a 1% HCl aqueous solution and 30 g of Zn were put into a 1000 mL beaker and the resulting mixture was stirred, 450 mL of 3% HCl and mercury (II) chloride (HgCl₂, 0.9 g) were added thereto and the resulting mixture was vigorously stirred at room temperature. The prepared zinc amalgam was washed using water and 1,4-dioxane, and then was added to a solution of the compound of Chemical Formula B (3 g, 19.46 mmol) and a solvent 1,4-dioxane (300 mL), and the resulting mixture was stirred for 20 minutes. The reaction mixture was cooled to 0° C., a 36% HCl aqueous solution (12 mL) was slowly added thereto, and the resulting mixture was stirred. The mixture was filtered using 200 mL of water, and distilled with 300 mL of ethyl acetate. The organic solvent layer was washed with water and a saturated NaCl aqueous solution, and then dried using MgSO₄. The solvent was removed by distillation under reduced pressure and the residue was separated using column chromatography (n-hexane: acetone = 8 : 1) to obtain a reddish-brown compound of Chemical Formula G (1.717 g, 67.4 %).

mp 162-163° C.; TLC Rf = 0.67 (n-hexane:acetone = 1:2); IR vₘₐₓ (cm¹) 3527, 3465, 3425, 2921, 2852, 1609, 1457, 1433, 1247, 1150; ¹H NMR (DMSO-d₆, 300 MHz) δ 8.62 (s, 2H, —OH), 7.76 (s, 1H, —OH), 5.92 (s, 1H, Ar—H), 1.86 (s, 6H, —CH₃); ¹³C NMR (DMSO-d₆, 150 MHz) δ 154.1 (2C), 153.2 (2C), 102.6 (1C), 94.6 (1C), 8.7 (2C).

### Reaction Step 9

The compound of Chemical Formula G (1.717 g, 11.14 mmol) and potassium carbonate (K₂CO₃, 6.1573 g, 44.55 mmol) were put into dry acetone and dissolved, then dimethyl sulfate (DMS 4.2154 ml, 44.55 mmol) was injected, and the resulting mixture was refluxed overnight. The mixture was cooled to room temperature, diluted with 300 mL of ethyl acetate, and then washed with a 1% HCl aqueous solution. The organic solvent layer was washed using water and a saturated NaCl aqueous solution, and dried using MgSO₄. The solvent was removed by distillation under reduced pressure and the residue was separated using column chromatography (n-hexane : acetone = 300 : 1) to obtain a white compound of Chemical Formula H (2.1550 g, 98.6%).

mp 152-155° C.; TLC R_{f} = 0.69 (n-hexane:acetone = 3:2); IR vₘₐₓ (cm¹) 2926, 1608, 1496, 1464, 1435, 1401, 1321, 1219, 1193, 1127; ¹H NMR (DMSO-d₆, 300 MHz) δ 6.41 (s, 1H, Ar—H), 3.77 (s, 6H, —OCH₃), 3.57 (s, 3H, —OCH₃), 1.98 (s, 6H, —CH₃); ¹³C NMR (DMSO-d₆, 150 MHz) δ 157.2 (1C), 156.3 (2C), 109.9 (2C), 91.8 (1C), 59.7 (1C), 55.5 (2C), 8.5 (2C).

### Reaction Step 10

Acetic anhydride (2.07 mL, 21.96 mmol) was injected into the compound of Chemical Formula H (2.1550 g, 10.98 mmol). The solution temperature was lowered to 0° C., and boron-trifluoride diethyl etherate (BF₃Et₂O, 2.7582 mL, 21.96 mmol) was added thereto. The resulting mixture was stirred for 3 hours while maintaining the reaction temperature at 90° C. Thereafter, the reaction product was cooled to room temperature and diluted with 300 mL of ethyl acetate. Then, the organic solvent layer was washed using a 1% HCl aqueous solution, water and a saturated NaCl aqueous solution, and dried using MgSO₄. The solvent was removed by distillation under reduced pressure and the residue was separated using column chromatography (n-hexane) to obtain a compound of Chemical Formula I (1.8812 g, 76.4%) as a yellow solid.

mp 48-50° C.; TLC R_{f}= 0.66 (n-hexane:acetone = 3:2); IR vₘₐₓ (cm⁻¹) 3440, 2941, 1621, 1454, 1417, 1364, 1318, 1283, 1198, 1171, 1120; ¹H NMR (DMSO-d₆, 300 MHz) δ 12.80 (s, 1H, —OH), 3.71 (s, 3H, —OCH₃), 3.69 (s, 3H, —OCH₃), 2.65 (s, 3H,—COCH₃), 2.09 (s, 3H, —CH₃), 2.03 (s, 3H, —CH₃); ¹³C NMR (DMSO-d₆, 150 MHz) 8203.8 (1C), 163.2 (1C), 158.8 (1C), 156.7 (1C), 115.4 (1C), 114.8 (1C), 113.9 (1C), 61.6 (1C), 59.9 (1C), 31.4 (1C), 8.7 (2C).

### Reaction Step 11

The compound of Chemical Formula I (1.8812 g, 8.389 mmol) and potassium carbonate (K₂CO₃, 1.3913 g, 10.06 mmol) were put into dry acetone and dissolved, then dimethyl sulfate (DMS mL, 10.06 mmol) was injected, and the resulting mixture was refluxed overnight. The reaction mixture was cooled at room temperature, diluted with 300 mL of ethyl acetate, and then washed with a 1% HCl aqueous solution. The organic solvent layer was washed using water and a saturated NaCl aqueous solution, and moisture was removed using MgSO₄. The solvent was removed by distillation under reduced pressure and the residue was separated using column chromatography (n-hexane : acetone = 300 : 1) to obtain a compound of Chemical Formula J (1.933 g, 96.7%) as a white solid.

¹H NMR (300 MHz, DMSO-d₆) δ 3.71 (s, 3H, —OCH₃), 3.69 (s, 6H, —OCH₃), 2.66 (s, 3H, —COCH₃), 2.10 (s, 3H, —CH₃), 2.04 (s, 3H, —CH₃)

### Reaction Step 12

After the compound of Chemical Formula J (0.25 g, 1.0492 mmol) was dissolved in 20 mL of methanol, appropriately substituted benzaldehyde (1.259 mmol) was added thereto. After potassium hydroxide (KOH, 0.1761 g, 3.1471 mmol) dissolved in 10 mL of methanol was added to this solution, the resulting solution was stirred for 48 hours. The reaction product was diluted with 150 mL of ethyl acetate and neutralized with 100 mL of an NH₄Cl aqueous solution. The organic solvent layer was washed using water and a saturated NaCl aqueous solution, and moisture was removed using MgSO₄. The solvent was removed by distillation under reduced pressure and the residue was separated using column chromatography (n-hexane or n-hexane : acetone = 300 : 1) to obtain compounds of Chemical Formulae 16 to 23.

[Compound of Chemical Formula 16 (1-(3′,5′-dimethyl-2′,4′,6′-trimethoxyphenyl)-3-(4-fluorophenyl)-2-propen-1-one)] Yield: 93.1%

¹H NMR (300 MHz, DMSO-d₆) δ 7.34-7.26 (d, J = 16.17 Hz, 1H, —C═C—H), 7.11-7.05 (d, J = 16.13 Hz, 1H, —C═C—H), 7.87-7.2 (m, 2H, Ar—H), 7.28-7.18 (m, 2H, Ar—H), 3.69 (s, 3H, —OCH₃), 3.58 (s, 6H, —OCH₃), and 2.13 (s, 6H, —CH₃); and ¹³C NMR (151 MHz, dmso) δ 194.36(1C), 164.72(1C), 163.06(1C), 159.07(1C), 154.24(1C), 144.17(1C), 131.56(1C), 131.50(1C), 131.20(1C), 131.18(1C), 128.75(1C), 125.03(1C), 120.51(2C), 116.49(1C), 116.35(1C), 62.00(1C), 60.17(1C), 9.59(2C).

[Compound of Chemical Formula 17 (1-(3′,5′-dimethyl-2′,4′,6′-trimethoxyphenyl)-3-phenyl-2-propen-1-one)] Yield: 96.31%

¹H NMR (300 MHz, DMSO-d₆) δ 7.32-7.24 (d, J = 16.17 Hz, 1H, —C═C—H), 7.14-7.05 (d, J= 16.11 Hz, 1H, —C═C—H), 7.74-7.67 (m, 2H, Ar—H), 7.46-7.35 (m, 3H, Ar—H), 3.70 (s, 3H, —OCH₃), 3.58 (s, 6H, —OCH₃), and 2.13 (s, 6H, —CH₃); and ¹³C NMR (151 MHz, dmso) δ 194.45(1C), 159.08(1C), 154.25(1C), 145.46(1C), 134.50(1C), 131.20(1C), 129.42(2C), 129.14(2C), 128.85(1C), 125.02(1C), 120.52(2C), 70.22(1C), 62.02(2C), 60.20(1C), 9.61(2C).

[Compound of Chemical Formula 18 (1-(3′,5′-dimethyl-2′,4′,6′-trimethoxyphenyl)-3-(4-tolyl)-2-propen-1-one)] Yield: 92.1%

¹H NMR (300 MHz, DMSO-d₆) δ 7.28-7.21 (d, J = 16.19 Hz, 1H, —C═C—H), 7.07-7.00 (d, J = 16.12 Hz, 1H, —C═C—H), 7.61-7.55 (d, J = 8.23 Hz, 2H, Ar—H), 7.24-7.17 (d, J = 8.00 Hz, 2H, Ar—H), 3.70 (s, 3H, —OCH₃), 3.58 (s, 6H, —OCH₃), 2.32 (s, 3H, —CH₃), and 2.13 (s, 6H, —CH₃); and ¹³C NMR (151 MHz, dmso) δ 194.45(1C), 159.00(1C), 154.19(2C), 145.62(1C), 141.31(1C), 131.77(1C) 130.05(2C), 129.15(2C), 127.95(1C), 125.11(1C), 120.49(2C), 61.99(2C), 60.18(1C), 21.48(1C), 9.6(2C).

[Compound of Chemical Formula 19 (1-(3′,5′-dimethyl-2′,4′,6′-trimethoxyphenyl)-3-(4-isopropylphenyl)-2-propen-1-one)] Yield: 94.3%

¹H NMR (300 MHz, DMSO-d₆) δ 7.29-7.22 (d, J = 16.17 Hz, 1H, —C═C—H), 7.10-6.95 (d, J = 16.12 Hz, 1H, —C═C—H), 7.65-7.58 (d, J = 8.20 Hz, 2H, Ar—H), 7.29-7.25 (d, J = 8.10 Hz, 2H, Ar—H), 3.70 (s, 3H, —OCH₃), 3.58 (s, 6H, —OCH₃), 3.00-2.80 (septet, J = 6.96 Hz, 1H, —CH), 2.13 (s, 3H, —CH₃), 1.20 (s, 3H, —CH₃), and 1.18 (s, 3H, —CH₃); and ¹³C NMR (151 MHz, dmso) δ 194.40(1C), 159.01(1C), 154.22(2C), 152.04(1C), 145.53(1C), 132.19(1C), 129.28(2C), 128.04(1C), 127.41(2C), 125.10(1C), 120.48(2C), 61.98(2C), 60.19(1C), 33.82(1C), 24.00(2C), 9.61(2C).

[Compound of Chemical Formula 20 (1-(3′,5′-dimethyl-2′,4′,6′-trimethoxyphenyl)-3-(4-methoxymethoxyphenyl)-2-propen-1-one)] Yield: 92.9%

¹H NMR (300 MHz, DMSO-d₆) δ 7.27-7.17 (d, J = 16.11 Hz, 1H, —C═C—H), 7.02-6.93 (d, J = 16.23 Hz, 1H, —C═C—H), 7.70-7.62 (d, J = 8.79 Hz, 2H, Ar—H), 7.06-7.0 (d, J = 8.74 Hz, 2H, Ar—H), 5.23 (2H, —CH2—O—), 3.70 (s, 3H, —OCH₃), 3.58 (s, 6H, —OCH₃), 3.37 (s, 3H, —OCH₃), and 2.13 (s, 6H, —CH₃); and ¹³C NMR (151 MHz, dmso) δ 194.33(1C), 159.29(1C), 158.92(1C), 154.16(2C), 145.35(1C), 130.92(2C), 128.03(1C), 127.09(1C), 125.21(1C), 120.46(2C), 116.83(2C), 94.12(1C), 61.97(2C), 60.18(1C), 56.18(1C), 9.61(2C).

[Compound of Chemical Formula 21 (1-(3′,5′-dimethyl-2′,4′,6′-trimethoxyphenyl)-3-(4-methoxyphenyl)-2-propen-1-one)] Yield: 91.8%

¹H NMR (300 MHz, DMSO-d₆) δ 7.27-7.13 (d, J = 16.10 Hz, 1H, —C═C—H), 7.02-6.88 (d, J = 15.90 Hz, 1H, —C═C—H), 7.70-7.60 (d, J = 8.70 Hz, 2H, Ar—H), 7.00-6.93 (d, J = 8.79 Hz, 2H, Ar—H), 3.79 (s, 3H, —OCH₃), 3.69 (s, 3H, —OCH₃), 3.58 (s, 6H, —OCH₃), and 2.13 (s, 6H, —CH₃); and ¹³C NMR (151 MHz, dmso) δ 194.33(1C), 161.88(1C), 158.88(1C), 154.14(2C), 145.58(1C), 131.03(2C), 127.05(1C), 126.66(1C), 125.27(1C), 120.45(2C), 114.92(2C), 61.96(2C), 60.18(1C), 55.81(1C), 9.61(2C).

[Compound of Chemical Formula 22 (1-(3′,5′-dimethyl-2′,4′,6′-trimethoxyphenyl)-3-(4-hydroxyphenyl)-2-propen-1-one)] Yield: 88.1%

¹H NMR (300 MHz, DMSO-d₆) δ 10.09 (s, 1H, —OH), 7.20-7.10 (d, J = 16.07 Hz, 1H, —C═C—H), 6.91-6.83 (d, J = 16.01 Hz, 1H, —C═C—H), 7.56-7.48 (d, J = 8.69 Hz, 2H, Ar—H), 6.80-6.74 (d, J = 8.62 Hz, 2H, Ar—H), 3.69 (s, 3H, —OCH₃), 3.57 (s, 6H, —OCH₃), and 2.12 (s, 6H, —CH₃); and ¹³C NMR (151 MHz, dmso) δ 194.26(1C), 160.65(2C), 158.81(1C), 154.12(2C), 146.14(1C), 131.22(2C), 125.71(1C), 125.34(1C), 120.41(2C), 116.32(2C), 61.93(2C), 60.17(1C), 9.59(2C).

[Compound of Chemical Formula 23 (1-(3′,5′-dimethyl-2′,4′,6′-trimethoxyphenyl)-3-(4-(N,N-dimethylamino)phenyl)-2-propen-1-one)] Yield: 78.3%

¹H NMR (300 MHz, DMSO-d₆) δ 7.16-7.06 (d, J = 15.94 Hz, 1H, —C═C—H), 6.83-6.75 (d, J = 15.90 Hz, 1H, —C═C—H), 7.52-7.43 (d, J = 8.90 Hz, 2H, Ar—H), 6.71-6.63 (d, J = 8.94 Hz, 2H, Ar—H), 3.69 (s, 3H, —OCH₃), 3.57 (s, 6H, —OCH₃), 2.98 (s, 6H, —NCH₃), and 2.12 (s, 6H, —CH₃); and ¹³C NMR (151 MHz, dmso) δ 193.91(1C), 158.60(1C), 154.04(2C), 152.44(1C), 146.83(1C), 130.91(2C), 125.69(1C), 123.61(2C), 121.58(1C), 120.34(2C), 112.20(2C), 111.31(1C), 61.89(2C), 60.17(1C), 9.61(2C).

### Example 2: Confirmation of Cytotoxicity of DMC Derivatives

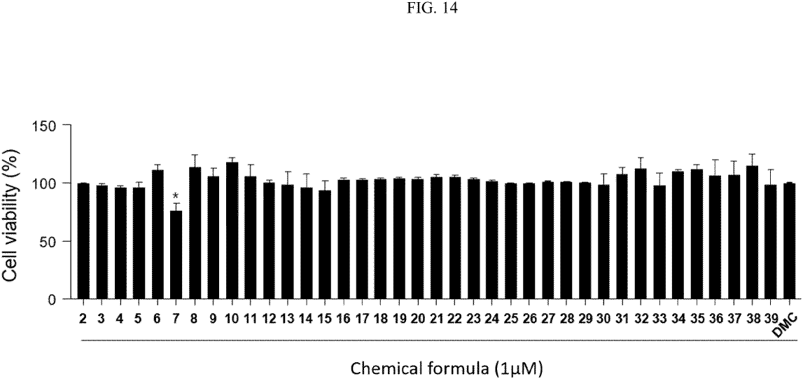

To check whether the DMC derivatives of Example 1 were cytotoxic, C2C12 cells were treated with each derivative at a concentration of 1 µM. More specifically, C2C12 myoblasts were primarily cultured in Dulbecco’s modified Eagle’s medium (DMEM) supplemented with 10% fetal bovine serum (FBS), and then cultured in DMEM supplemented with 2% horse serum for 5 days to differentiate into myotubes. After the cells were then treated with DMC derivatives and cultured for 24 hours, cytotoxicity was measured using a Cell Counting Kit-8 (CCK-8) assay kit (Dojindo Laboratories). As a negative control (Veh), the cells were treated with 0.1% DMSO. As a result, the DMC derivatives except for Chemical Formula 7 did not affect cell growth at all at a concentration of 1 µM, and most of the cells showed a viability of 90% or more (FIG. 14). From this, it could be confirmed that the DMC derivatives according to the present invention did not significantly affect cell viability.

### Example 3: Confirmation of Effect of DMC Derivatives on PPARγ Activity

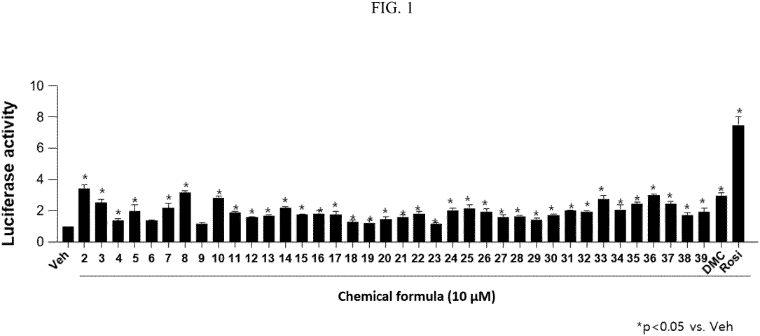

Peroxisome proliferator-activated receptor gamma (PPARγ) is a ligand-activated transcription factor, and is known to play a very important role in regulating energy homeostasis or inflammatory responses. Moreover, PPARγ agonists, which activate PPARγ, cause adipocytes to differentiate, take in blood free fatty acids and accumulate the blood free fatty acids in subcutaneous fat, thereby not only lowering blood free fatty acids to improve insulin resistance, but also increasing the expression of glucose transporters to help glucose uptake by the liver and skeletal muscle. In addition, PPARγ agonists are known to show the effect of lowering blood pressure in patients exhibiting insulin resistance and exhibit the effect of ameliorating vascular endothelial cell-dependent vasodilation, and the like, thereby being able to effectively treat metabolic diseases. Therefore, a luciferase assay system (Promega) was used in order to confirm the effect of the DMC derivatives of Example 1 on PPARγ activity. More specifically, Cos7 cells were transfected with 0.1 µg of a plasmid carrying a pPPRE-tk-luc reporter and 0.1 µg of a PPARγ overexpression vector. Then, the transfected Cos7 cells were treated with 10 µM of each of the DMC derivatives, and then cultured for 24 hours. The cells were treated with 10 µM rosiglitazone (Rosi) as a positive control. Next, after 24 hours, the activity of PPARγ was measured using luciferase activity. Thereafter, all experiments were repeated at least three times, and the results were expressed as mean ± standard error. Statistical significance was confirmed by a Student’s t-test, and when P < 0.05, it was determined to be statistically significant. The results are shown in FIG. 1.

As shown in FIG. 1, it was confirmed that the DMC derivatives according to the present invention can significantly activate PPARγ. Particularly, Chemical Formulae 2, 3, 7, 8, 10, 14, 24, 25, 31, 33, 34, 35, 36, and 37 significantly increased the activity of PPARγ by 2-fold or more.

From the above results, it can be confirmed that the DMC derivatives of the present invention exhibit activity as agents which increase the activity of PPARγ, and can be used as insulin sensitizers that increase insulin sensitivity, therapeutic agents for metabolic diseases, and the like.

### Example 4: Confirmation of Effect of DMC Derivatives on AMPK Activity

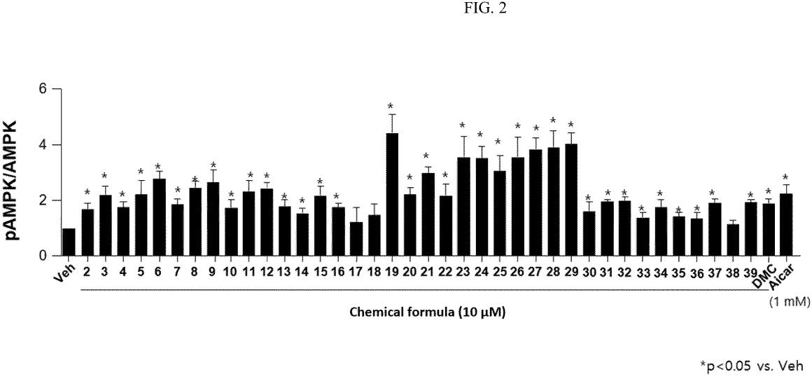

AMP-activated protein kinase (AMPK) is a member of serine/threonine kinases, and is an enzyme known as an energy sensor which senses the intracellular energy state. Moreover, the activation of AMPK is known to show the effect of increasing glucose transport and promoting fatty acid oxidation in skeletal muscle, and to increase the synthesis of cholesterol and triglycerides, decrease lipogenesis, and increase fatty acid oxidation, and ketogenesis, and the like in the liver. Further, the activation of AMPK is known to reduce adipogenesis in adipose tissue and increase insulin secretion in pancreatic beta cells, thereby being able to be applied to the treatment of various metabolic diseases. Therefore, western blotting was performed in order to confirm the effect of the DMC derivatives of Example 1 on AMPK activity. More specifically, the C2C12 myoblasts were primarily cultured in DMEM supplemented with 10% fetal bovine serum, and then cultured in DMEM supplemented with 2% horse serum for 5 days to differentiate into myotubes. Then, the differentiated myotubes were treated with 10 µM DMC derivative and cultured for 24 hours, and then cells were collected, and disrupted using an ultrasonic disperser to obtain proteins. Next, western blotting was performed using a pAMPK antibody (Cell Signaling Technology) and an AMPK antibody (Cell Signaling Technology) to measure the amount of phosphorylated AMPK. 1 mM AICAR was used as a positive control, and 20 µg of protein was used for each sample in western blotting. The results are shown in FIG. 2.

As shown in FIG. 2, it was confirmed that AICAR, which was used as a positive control, and DMC increased the activity of AMPK. The DMC derivatives according to the present invention increased the activity of AMPK. Particularly, the DMC derivatives of Chemical Formulae 3, 5, 6, 8, 9, 11, 12, 15, 19, 20, 21, 22, 23, 24, 25, 26, 27, 28, 29, 31, 32, 37, and 39 further increased the activity of AMPK similarly to or more effectively than the positive controls.

From the above results, it could be confirmed that the DMC derivatives of the present invention can be used as drugs which increase the activity of AMPK.

### Example 5: Confirmation of Effect of DMC Derivatives on Fatty Acid Oxidation

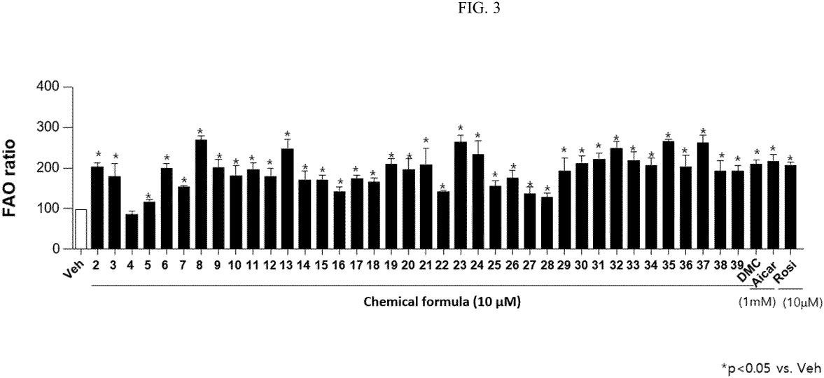

It is known that the risk of metabolic and cardiovascular diseases increases due to an increase in triglycerides and a decrease in high-density cholesterol when fatty acid metabolism is abnormal. Therefore, it was checked whether the DMC derivatives of Example 1 affect fatty acid oxidation. For the measurement of a fatty acid oxidation rate, the C2C12 myoblasts were primarily cultured in DMEM supplemented with 10% fetal bovine serum, and then cultured in DMEM supplemented with 2% horse serum for 5 days to differentiate into myotubes. Then, the differentiated myotubes were treated with 10 µM DMC derivative and cultured for 24 hours, and then cells were obtained. Next, the obtained cells were lysed using a mitochondrial isolation buffer (250 mmol/L sucrose, 10 mmol/L Tris-HCl, and 1 mmol/LEDTA), and then treated with 0.2 mmol/L [1-¹⁴C]palmitate and reacted for 3 hours. Then, the amount of ¹⁴CO₂ generated at this time was measured, and the measured amount of ¹⁴CO₂ was corrected with the total protein amount to compare the fatty acid oxidation rate. The results are shown in FIG. 3.

As shown in FIG. 3, it was confirmed that the DMC derivatives according to the present invention induce fatty acid oxidation. Particularly, the DMC derivatives of Chemical Formulae 2, 3, 6, 8, 9, 10, 11, 12, 13, 19, 20, 21, 23, 24, 29, 30, 31, 32, 33, 34, 35, 36, 37, 38, and 39 exhibited fatty acid oxidation rates similar to those of AICAR and Rosi, which were used as positive controls, and DMC.

From the above results, it could be confirmed that the DMC derivatives of the present invention can effectively suppress the progression of metabolic diseases such as diabetes by increasing fatty acid oxidation.

### Example 6: Confirmation of Effect of DMC Derivatives on Migration of Vascular Smooth Muscle Cells

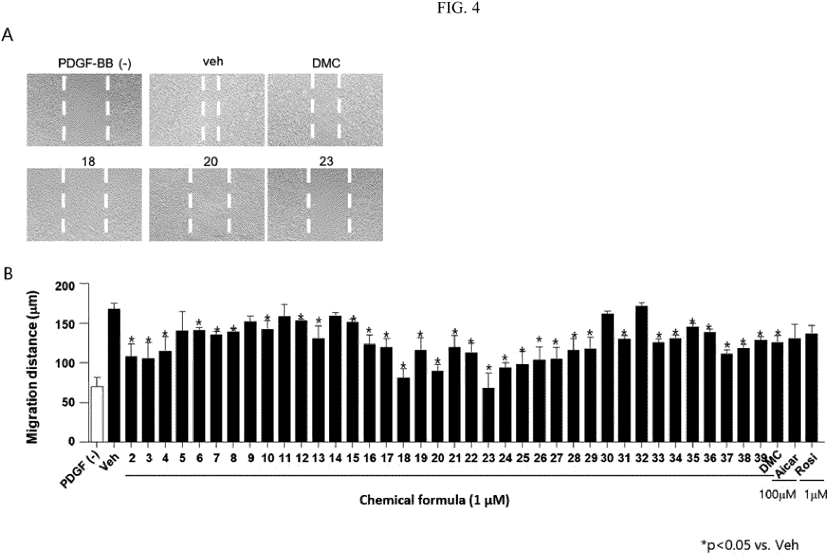

Proliferation and migration of vascular smooth muscle cells are known to be essential steps in the progression of vascular diseases such as restenosis and arteriosclerosis. Therefore, in order to confirm the effect of the DMC derivatives of Example 1 on the migration of rat aortic smooth muscle cells, smooth muscle cells were treated with 10 ng/mL platelet-derived growth factor (PDGF) and 1 µM DMC derivative and cultured. Then, the degree of cell migration was measured. The results are shown in FIG. 4.

As shown in FIG. 4, it was confirmed that the DMC derivatives according to the present invention suppress the migration of vascular smooth muscle cells. Particularly, the DMC derivatives of Chemical Formulae 2, 3, 4, 13, 15, 16, 17, 18, 19, 20, 21, 22, 23, 24, 25, 26, 27, 28, 29, 31, 33, 34, 37, 38, and 39 similarly or more effectively suppressed cell migration by PDGF than AICAR and Rosi, which were used as positive controls, and DMC.

From the above results, it could be confirmed that the DMC derivatives of the present invention can effectively suppress vascular diseases caused by metabolic diseases such as arteriosclerosis by suppressing the migration of vascular smooth muscle cells to the tunica intima.

### Example 7: Confirmation of Effect of DMC Derivatives on Binding Between Vascular Endothelial Cells and Immune Cells

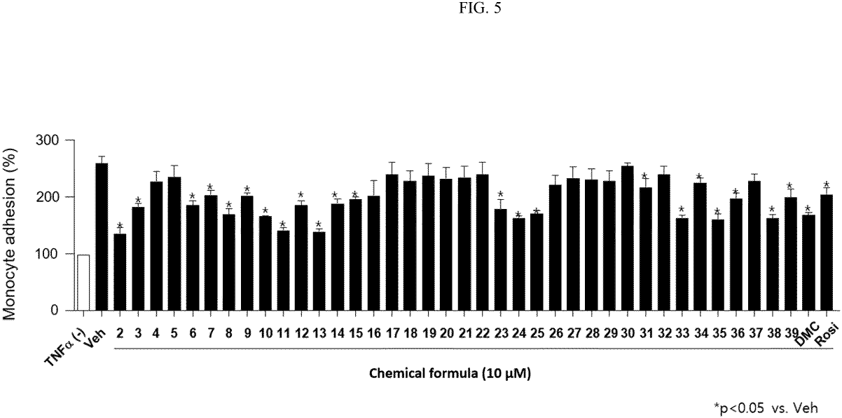

In the early stages of progression of vascular diseases such as arteriosclerosis, adhesion materials are expressed in vascular endothelial cells due to various environmental factors, thereby increasing the binding of immune cells. Therefore, in order to check whether the DMC derivatives of Example 1 affect the binding between vascular endothelial cells and immune cells, vascular endothelial cells (HUVECs) were treated with 10 ng/mL TNFα and 10 µM DMC derivative and cultured for 24 hours. Then, THP-1 (human monocyte) immune cells labeled with a fluorescent protein were added to the culture solution, and the cells were additionally cultured for 1 hour. After the culture was completed, unbound immune cells were removed using a phosphate buffer solution, and then the degree of binding of immune cells to vascular endothelial cells was determined by measuring the intensity of fluorescence. The results are shown in FIG. 5.

As shown in FIG. 5, it was confirmed that the DMC derivatives according to the present invention suppress the binding of immune cells to vascular endothelial cells. Particularly, the DMC derivatives of Chemical Formula 2, 3, 6, 7, 8, 9, 10, 11, 12, 13, 14, 15, 23, 24, 25, 33, 35, 36, 38, and 39 similarly or more effectively suppressed the binding of immune cells than Rosi, which was used as a positive control.

From the above results, it could be confirmed that the DMC derivatives of the present invention can suppress the initial occurrence of vascular diseases caused by metabolic diseases such as arteriosclerosis by suppressing the binding of immune cells to vascular endothelial cells.

### Example 8: Confirmation of Antioxidant Effect of DMC Derivatives

NF-E2-related factor 2 (Nrf2), which is a transcription factor, plays an important role in maintaining the homeostasis of cells by regulating the basal expression levels or induced expression levels of proteins which suppress oxidation when cells are exposed to chemicals or oxidative stress, or proteins related to the transport of detoxification enzymes or xenobiotics. In addition, Nrf2 is also known to be involved in inflammatory responses, lipid synthesis, and the like. Kelch-like ECH-associated protein 1 (Keap 1) binds to Nrf2 to induce the Nrf2 protein degradation process by proteasomes. Conversely, it is known that Nrf2 activators separate Nrf2 from Keap1, and the separated Nrf2 translocates to the nucleus to increase the expression of antioxidant proteins such as NQO1 and HO1, thereby effectively suppressing obesity, non-alcoholic fatty liver, diabetic complications, and the like. Therefore, it was checked whether the DMC derivatives of Example 1 exhibit an antioxidant effect.

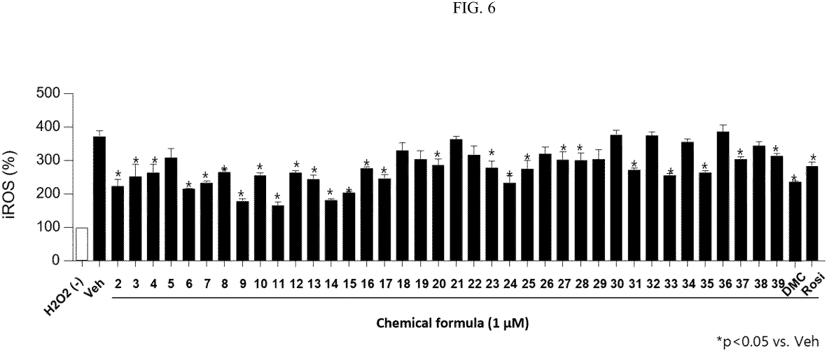

First, the antioxidant effect was checked using kidney cells. More specifically, a human kidney proximal tubule epithelial cell (HK-2) cell line was treated with 1 µM DMC derivative and cultured for 18 hours. Then, 500 µM H₂O₂ was added to the culture medium and the culture medium was incubated for 6 hours. Thereafter, the amount of intracellular reactive oxygen species (iROS) was measured using dichlorohydro-fluorescein diacetate (DCF-DA). The results are shown in FIG. 6.

As shown in FIG. 6, it was confirmed that the DMC derivatives according to the present invention decrease the amount of reactive oxygen species (ROS). Particularly, the DMC derivatives of Chemical Formulae 2, 3, 4, 6, 7, 8, 9, 10, 11, 12, 13, 14, 15, 16, 17, 20, 23, 24, 25, 31, 33, and 35 effectively reduced ROS.

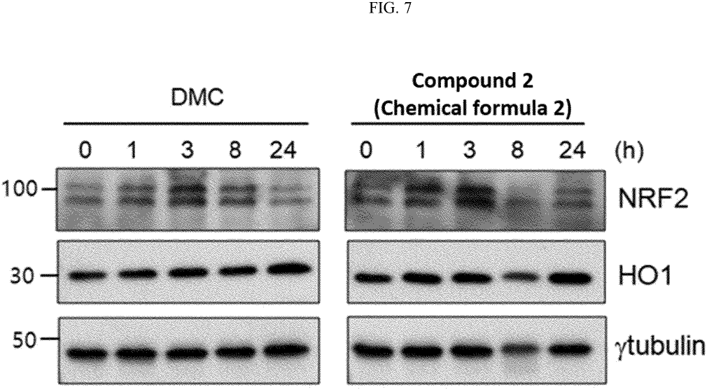

In addition, to check whether the Nrf2 protein was increased, a HK-2 cell line was treated with 1 µM DMC derivative and cultured for 24 hours. Then, after culturing, the cells were collected at 1, 3, 8 and 24 hours, respectively, and western blotting was performed. The results are shown in FIG. 7.

As shown in FIG. 7, it was confirmed that the expression level of Nrf2 in kidney cells was increased the most at 3 hours after treatment and decreased thereafter.

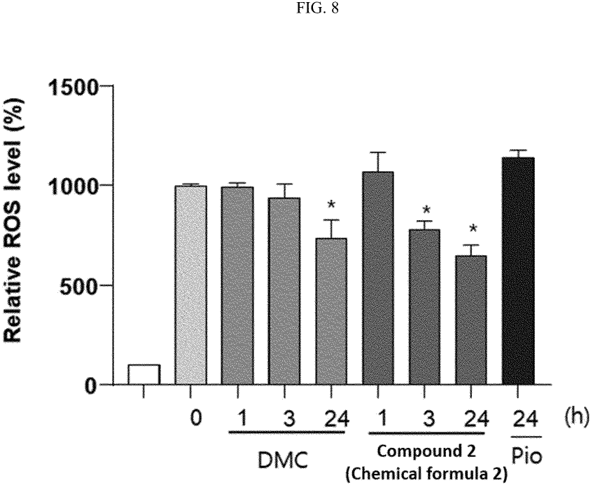

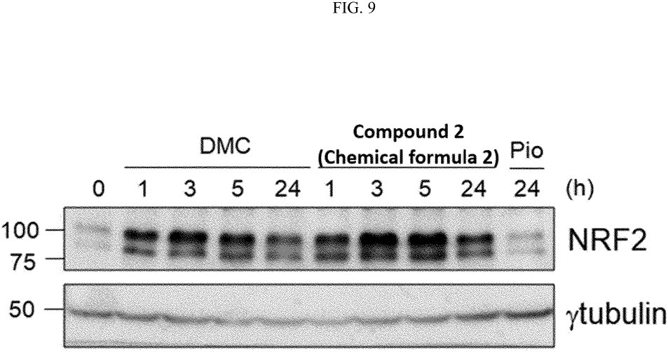

Second, HepG2 cells, which are human-derived hepatocytes, were treated with 1 µM of DMC or the compound of Chemical Formula 2, respectively, and then the amount of intracellular reactive oxygen species and the expression level of Nrf2 protein were confirmed by the same method. Pioglitazone (Pio) was used as a control. The results are shown in FIGS. 8 and 9, respectively.

As shown in FIGS. 8 and 9, it was confirmed that the amount of Nrf2 protein in hepatocytes was increased up to 5 hours after treatment, and the amount of ROS was also significantly decreased after 3 hours when the amount of Nrf2 protein was increased.

From the above results, it could be confirmed that the DMC derivatives of the present invention exhibited an antioxidant effect by increasing the expression level of Nrf2 protein in the kidneys and liver, through which, it could be confirmed that the DMC derivatives could be used to treat various metabolic diseases.

### Example 9: Confirmation of in Vivo Therapeutic Effect of DMC Derivatives

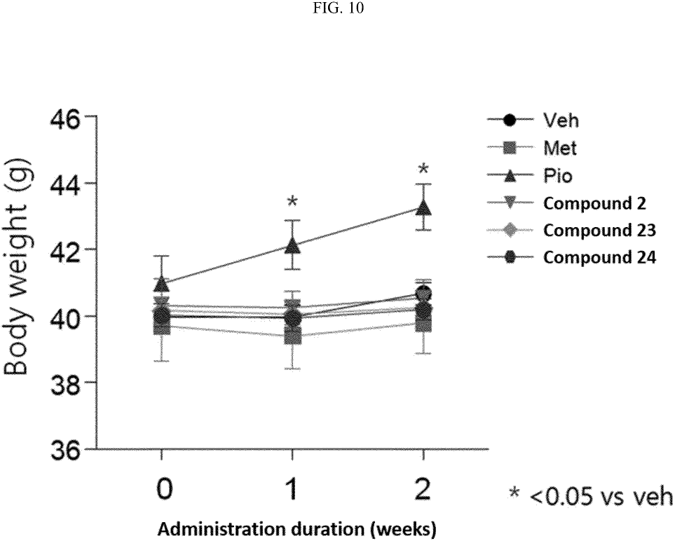

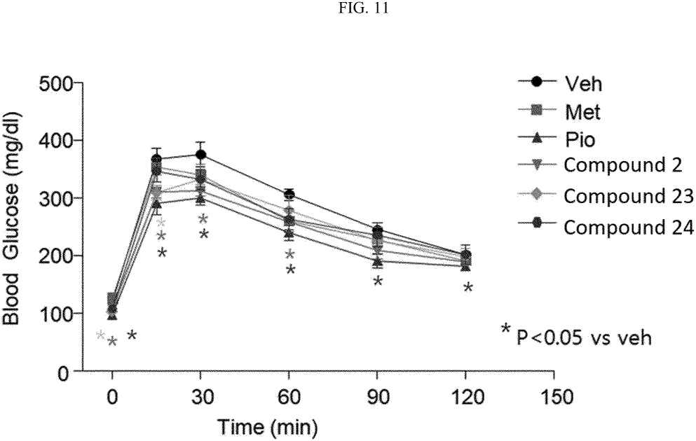

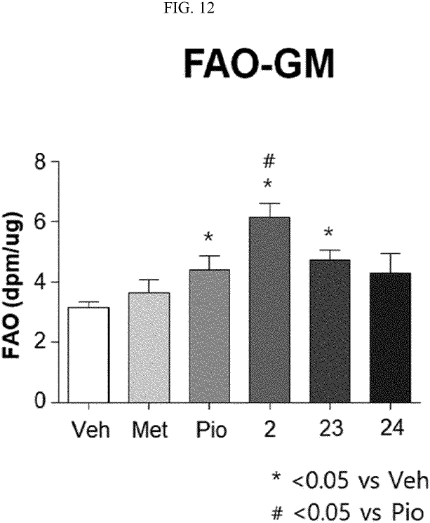

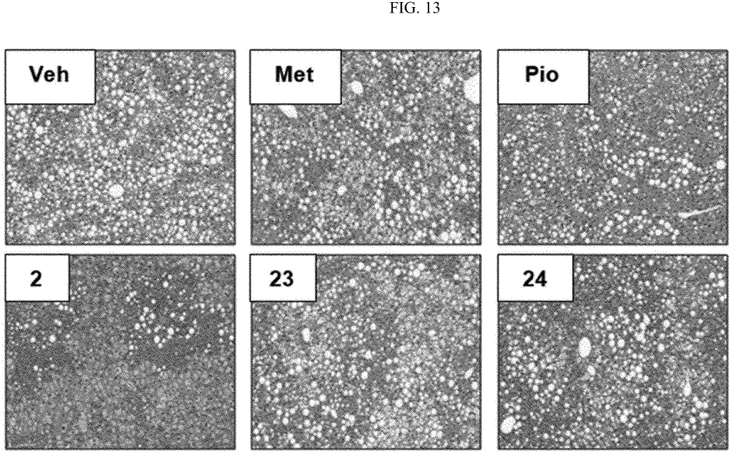

In order to check whether the DMC derivatives of the present invention also exhibit a therapeutic effect on metabolic diseases in vivo, an experiment was performed using a diet-induced obese (DIO) mouse model through a high-fat diet. DMC derivatives of the present invention (30 mg/kg/day), pioglitazone (30 mg/kg/day), and metformin (Met) (200 mg/kg/day) were orally administered to 8-week-old DIO mice daily for 3 weeks, two weeks after oral administration, an intraperitoneal glucose tolerance test (IPGTT) was performed, and three weeks later, the mice were euthanized, and then experiments were performed after obtaining tissues. The same dose of phosphate buffer solution was administered as a negative control (Veh). Experiments were performed using 6 to 7 mice per experimental group, and the guidelines for the care, use, treatment and management of all experimental animals were approved by the Institutional Animal Care and Use Committee (IACUC) of Seoul National University Hospital. The body weights of the mice according to the administration period were measured at weekly intervals, and the results are shown in FIG. 10. Glucose tolerance test results are shown in FIG. 11, and the results of measuring a fatty acid oxidation rate (FAO) using gastrocnemius muscle (GM) obtained after euthanasia are shown in FIG. 12. Then, Nile Red staining was performed using an obtained liver tissue to confirm the degree of fat accumulation in the liver tissue. The results are shown in FIG. 13.

As shown in FIG. 10, it was confirmed that in the experimental groups to which DMC derivatives of the present invention were administered, a body weight gain was not induced unlike pioglitazone-administrated group.

As shown in FIG. 11, it was confirmed that in the experimental groups to which DMC derivatives of the present invention were administered, fasting blood glucose was decreased and glucose tolerance was improved compared to the negative control.

As shown in FIG. 12, it was confirmed that in the experimental groups to which DMC derivatives of the present invention were administered, a fatty acid oxidation rate was increased compared to the negative control.

In addition, as shown in FIG. 13, it was confirmed that in the experimental groups to which DMC derivatives of the present invention were administered, fat accumulation in liver tissue was decreased compared to the negative control.

From the above results, it could be confirmed that the DMC derivatives of the present invention exhibited a similar or increased metabolic index improvement effect compared to the positive control using metformin and pioglitazone, which are used as therapeutic agents for metabolic diseases.

From the above results, it could be confirmed that the DMC derivatives of the present invention can not only reduce insulin resistance by increasing the activities of PPARγ and AMPK, but also exhibit a therapeutic effect on metabolic diseases such as hyperglycemia, obesity, diabetes, and non-alcoholic fatty liver by increasing fatty acid oxidation. Also, the DMC derivatives of the present invention can exhibit a therapeutic effect on metabolic diseases such as hyperlipidemia, arteriosclerosis, hypertension, and coronary arteriosclerosis, and cardiovascular diseases (stroke, angina pectoris, myocardial infarction, and peripheral vascular disease) induced by metabolic diseases by suppressing the migration of vascular smooth muscle cells to the tunica intima and the binding of vascular endothelial cells and immune cells to prevent blood vessels from narrowing. Furthermore, it could be confirmed that the DMC derivative of the present invention acted as an activator of Nrf2 to exhibit an antioxidant effect, thereby exhibiting a therapeutic effect on not only metabolic diseases but also complications caused by diabetes (diabetic retinopathy, diabetic nephropathy, diabetic foot disease, and diabetic neuropathy). Therefore, it could be confirmed that the DMC derivatives of the present invention can be widely applied to metabolic diseases with complex symptoms, or cardiovascular diseases and complications caused by metabolic diseases, and the like to efficiently manage and treat various metabolic diseases.

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described embodiments are only exemplary in all aspects and are not restrictive.

DMC derivatives of the present invention not only can reduce insulin resistance by increasing the activity of PPARγ and AMPK, but also exhibit an effect of preventing blood vessels from narrowing by increasing fatty acid oxidation and suppressing the migration of vascular smooth muscle cells to the tunica intima and the binding of vascular endothelial cells and immune cells. Furthermore, the DMC derivatives of the present invention act as an activator of Nrf2 to exhibit an antioxidant effect, and thus, can be effectively used for the treatment of various metabolic diseases such as obesity, diabetes, hypertension, hyperlipidemia, arteriosclerosis, coronary arteriosclerosis, non-alcoholic fatty liver, diabetic retinopathy, diabetic nephropathy, diabetic foot disease, diabetic neuropathy, stroke, angina pectoris, myocardial infarction, and peripheral vascular disease, complications caused by metabolic diseases, and the like.

What is claimed is:

1. A method of preventing, alleviating, or treating a metabolic disease comprising administering a composition comprising a derivative of 2′,4′-dihydroxy-6′-methoxy-3′,5′-dimethylchalcone represented by Chemical Formula 1, or a pharmaceutically acceptable salt thereof as an active ingredient to a subject in need thereof: 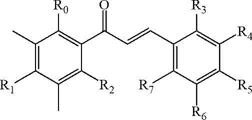
wherein:
R₀, R₁, and R₂ are each independently a hydroxy group (OH), a methoxymethoxy group (OCH₂OCH₃; OMOM), or a C1-C10 alkoxy group, wherein at least any one of R₀, R₁, and R₂ is not OH,
when R₀ and R₁ are simultaneously a methoxy group (OCH₃; OMe) or simultaneously OMOM, R₂ is not OH or OMOM,
R₃ to R₇ are each independently any one selected from the group consisting of a halogen element, hydrogen (H), deuterium (D), a hydroxyl group (OH), a thiol group (SH), an amino group (NH₂), a nitrile group, a nitro group, a substituted or unsubstituted C1-C10 alkylamino group, a substituted or unsubstituted C1-C10 alkoxy group, a substituted or unsubstituted C1-C10 alkylsulfoxy group, a substituted or unsubstituted C1-C10 alkyl group, a substituted or unsubstituted C2-C10 alkenyl, a substituted or unsubstituted C2-C10 alkynyl, a substituted or unsubstituted C6-C20 aryl group, a substituted or unsubstituted C1-C10 alkylthio group, and a substituted or unsubstituted C1-C10 alkylsulfonyl group,
wherein the ‘substituted or unsubstituted’ is unsubstituted or substituted with one or more substituents selected from the group consisting of a halogen group, a nitrile group, a nitro group, a hydroxy group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an alkynyl group, an aryl group, an aralkyl group, an aralkenyl group, an alkylaryl group, an alkylamine group, an aralkylamine group, a heteroarylamine group, an arylamine group, an arylphosphine group, and a heterocyclic group,
at least any one of R₃ to R₇ is not hydrogen when R₁ and R₂ are simultaneously a hydroxyl group, and
R₃ to R₇ are not a hydroxyl group when R₁ and R₂ are simultaneously a hydroxyl group.

2. The method of claim 1, wherein the derivative of 2′,4′-dihydroxy-6′-methoxy-3′,5′-dimethylchalcone represented by Chemical Formula 1 is any one or more compounds selected from the group consisting of Chemical Formulae 2 to 39, or a pharmaceutically acceptable salt thereof.

| | | | |
|---|---|---|---|
| Chemical formula 2 | Chemical formula 3 | Chemical formula 4 | Chemical formula 5 |
| 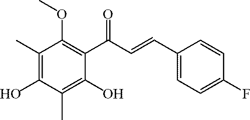 [SMILES: COc1c(C)c(O)c(C)c(O)c1C(=O)/C=C/c1ccc(F)cc1] | 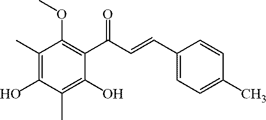 [SMILES: COc1c(C)c(O)c(C)c(O)c1C(=O)/C=C/c1ccc(C)cc1] | 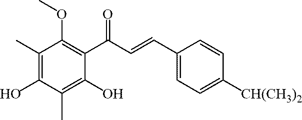 [SMILES: COc1c(C)c(O)c(C)c(O)c1C(=O)/C=C/c1ccc(C(C)C)cc1] | 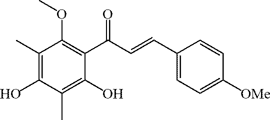 [SMILES: COc1ccc(/C=C/C(=O)c2c(O)c(C)c(O)c(C)c2OC)cc1] |
| Chemical formula 6 | Chemical formula 7 | Chemical formula 8 | Chemical formula 9 |
| 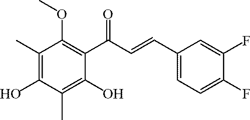 [SMILES: COc1c(C)c(O)c(C)c(O)c1C(=O)/C=C/c1ccc(F)c(F)c1] | 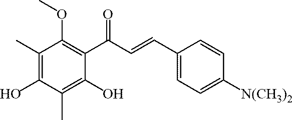 [SMILES: COc1c(C)c(O)c(C)c(O)c1C(=O)/C=C/c1ccc(N(C)C)cc1] | 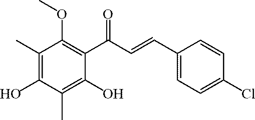 [SMILES: COc1c(C)c(O)c(C)c(O)c1C(=O)/C=C/c1ccc(Cl)cc1] | 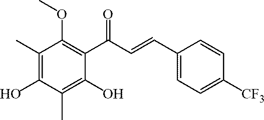 [SMILES: COc1c(C)c(O)c(C)c(O)c1C(=O)/C=C/c1ccc(C(F)(F)F)cc1] |
| Chemical formula 10 | Chemical formula 11 | Chemical formula 12 | Chemical formula 13 |
| 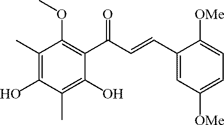 [SMILES: COc1ccc(OC)c(/C=C/C(=O)c2c(O)c(C)c(O)c(C)c2OC)c1] | 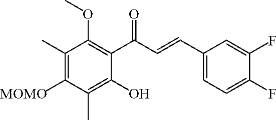 | 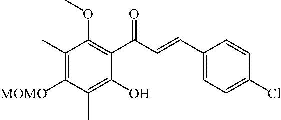 | 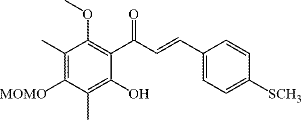 |
| Chemical formula 14 | Chemical formula 15 | Chemical formula 16 | Chemical formula 17 |
| 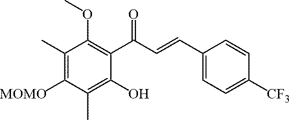 | 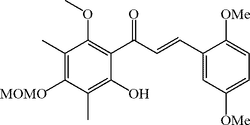 | 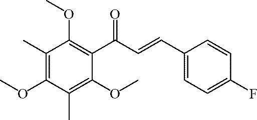 [SMILES: COc1c(C)c(OC)c(C(=O)/C=C/c2ccc(F)cc2)c(OC)c1C] | 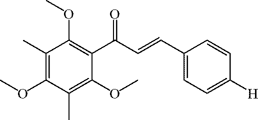 [SMILES: COc1c(C)c(OC)c(C(=O)/C=C/c2ccccc2)c(OC)c1C] |
| Chemical formula 18 | Chemical formula 19 | Chemical formula 20 | Chemical formula 21 |
| 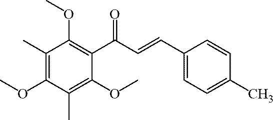 | 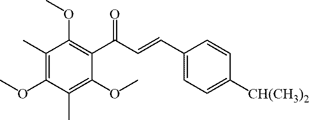 [SMILES: COc1c(C)c(OC)c(C(=O)/C=C/c2ccc(C(C)C)cc2)c(OC)c1C] | 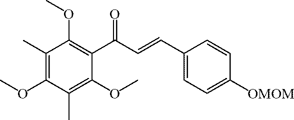 | 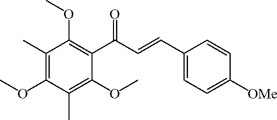 [SMILES: COc1ccc(/C=C/C(=O)c2c(OC)c(C)c(OC)c(C)c2OC)cc1] |
| Chemical formula 22 | Chemical formula 23 | Chemical formula 24 | Chemical formula 25 |
| 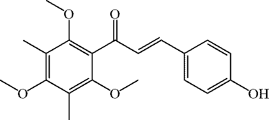 [SMILES: COc1c(C)c(OC)c(C(=O)/C=C/c2ccc(O)cc2)c(OC)c1C] | 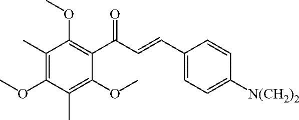 | 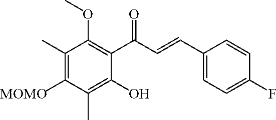 | 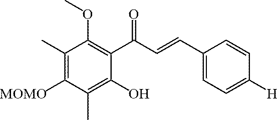 |
| Chemical formula 26 | Chemical formula 27 | Chemical formula 28 | Chemical formula 29 |
| 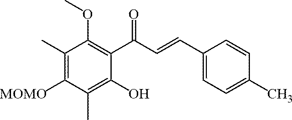 | 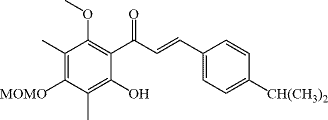 | 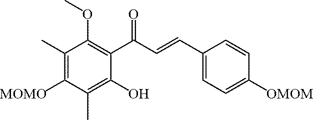 | 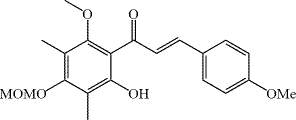 |
| Chemical formula 30 | Chemical formula 31 | Chemical formula 32 | Chemical formula 33 |
| 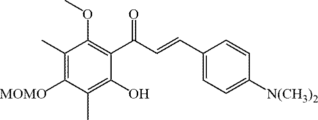 | 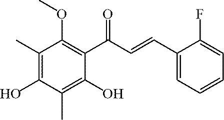 [SMILES: COc1c(C)c(O)c(C)c(O)c1C(=O)/C=C/c1ccccc1F] | 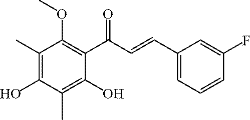 [SMILES: COc1c(C)c(O)c(C)c(O)c1C(=O)/C=C/c1cccc(F)c1] | 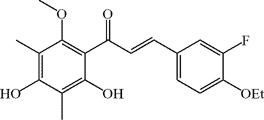 [SMILES: CCOc1ccc(/C=C/C(=O)c2c(O)c(C)c(O)c(C)c2OC)cc1F] |
| Chemical formula 34 | Chemical formula 35 | Chemical formula 36 | Chemical formula 37 |
| 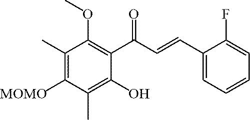 | 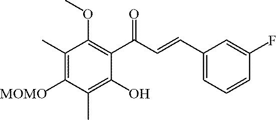 | 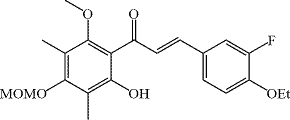 | 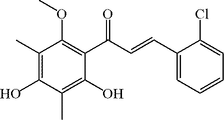 [SMILES: COc1c(C)c(O)c(C)c(O)c1C(=O)/C=C/c1ccccc1Cl] |
| Chemical formula 38 | Chemical formula 39 | | |
| 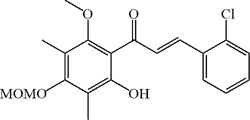 | 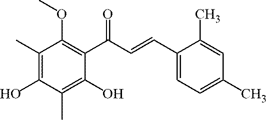 | | |

.

3. The method of claim 1, wherein the metabolic disease is any one or more selected from the group consisting of obesity, diabetes, hypertension, hyperlipidemia, arteriosclerosis, coronary arteriosclerosis, non-alcoholic fatty liver, diabetic retinopathy, diabetic nephropathy, diabetic foot disease, diabetic neuropathy, stroke, angina pectoris, myocardial infarction, and peripheral vascular disease.
